(19) United States
(12) Patent Application Publication
(10) Pub. No.: US 2023/0120124 A1
(43) Pub. Date: Apr. 20, 2023

(54) BALANCE TRAINER SIMULATOR SYSTEM AND METHOD

(71) Applicant: B. G. NEGEV TECHNOLOGIES AND APPLICATIONS LTD., AT BEN-GURION UNIVERSITY, Beer Sheva (IL)

(72) Inventors: Itshak MELZER, Lehavim (IL); Amir SHAPIRO, Meitar (IL); Shani BATCIR, Maagan Michael (IL); Koby LIVNE, Mezadot Yehuda (IL); Rotem LEV LEHMAN, Nir Banim (IL); Shmil EDELMAN, Beer Sheva (IL)

(21) Appl. No.: 17/909,656

(22) PCT Filed: Mar. 24, 2021
(86) PCT No.: PCT/IL2021/050328

§ 371 Date: Sep. 6, 2022

Related U.S. Application Data
(60) Provisional application No. 62/993,820, filed on Mar. 24, 2020.

(51) Int. Cl. A63B 69/00; A61B 5/11; A61B 5/00; A63B 21/005; A63B 22/06; A63B 22/16

(52) U.S. Cl. CPC A63B 69/0053; A61B 5/1116; A61B 5/1128; A61B 5/4023; A63B 21/0058; A63B 22/16; A63B 22/0605; A63B 26/003

(57) ABSTRACT

Provided herein is a mechatronic simulator system that provides various types of unexpected perturbations to challenge the proactive and reactive balance control in subjects to thereby improve balance control of the subject. The system includes a motion capture unit and a processing unit capable of analyzing the subject’s response to the perturbations, provide real time feedback, adjust and/or determine training sessions.

### TECHNICAL FIELD

The present disclosure relates generally to a balance trainer simulator system utilizing stationary bicycle that provide customized unexpected perturbations during use, and methods of using the same for improving balance control of a user.

### BACKGROUND

Balance control and particularly balance reactive responses that contribute to maintaining balance when balance is lost and preventing falls is impaired in older adults. This leads to increased risk of falls and injurious falls. Improving balance recovery responses is one of the goals in fall-prevention training programs.

The rising proportion of the elderly population and their associated-morbidity is placing upward pressure on overall health care resources (National Institute on Aging. Growing Older in America, 2007). One of the serious health problems are falls which is the leading cause of fatal and nonfatal injuries in this population. More than 30% of community-dwelling older persons fall at least once a year and about 50% among age of 80 years and older (CDC, 2016. Rubenstein, 2006). A range of 20-30%, of those who fell, suffer acute injuries, such as hip fractures and traumatic brain injuries, that reduce mobility, independence, and even resulting in a death (CDC 2106, Stevens JA., 2005). In 2015 in the US, the medical costs for older adult falls was above $50 billion (Florence CS et al. 2018). Balance control play a critical role in preventing falls and preserving functional independence (Stevens JA., 2005) specifically balance reactive strategies that evoked by unexpected perturbation of balance.

Ineffective balance reactive reactions following unexpected loss of balance is one of the major causes of falls in older adults living in the community (Maki, B.E., & McIlroy, 1997). Unexpected loss of balance, such as a slip or a trip, trigger automatic postural responses, which act to restore equilibrium (Nashner LM 1976, Nashner LM 1977, Nashner LM 1980). These balance reactive responses are specific to the size, type and direction of the perturbation (Nashner LM 1976, Nashner LM 1977, Nashner LM 1980). For example, fixed base-of-support strategies (feet remain in place) is used to restore balance by ankle, hip and trunk movements during minor to moderate perturbations, while in larger perturbations, the change of base-of-support strategies are used (Maki & McIlroy 1997). Systematic reviews published recently found that perturbation training programs are effective to improve balance recovery strategies as well as reduce fall incidence (McCrum et al., 2017; Gerards et al., 2017), even reduced diverse risks of falls and the rate of falls (Mansfield et al.,2015 Okubo et al., 2016).

Other research (Rissel et al., 2013; and Batcir et al., 2018) has found that older adults who are bicycling outdoors regularly have a better balance control than age-matched controls (Batcir et al., 2018) and the amount of outdoor bicycling were associated with better balance control (Rissel et al., 2013).

Perturbations training intervention programs are conducted by different mechatronic systems that provide external perturbations in standing and walking position in various ways. These training devices were designed to train specifically the change of support i.e., stepping reactions in older adults who were able to stand or walk independently without external support for whole training sessions, usually lasting 20-45 minutes each. Thus, older adults who unable to walk independently on a treadmill such as pre-frail or frail older adults as well as people with neurological disorders are less able to participate in these training programs. In order to match perturbation training approach for these people, designing and developing a mechatronic system that provide balance training that include perturbations while sitting can be valuable for older adults.

Perturbation training during standing or treadmill walking, that specifically challenge the reactive balance responses, exhibited beneficial effects, however only older adults that are able to walk independently can utilize this training regimes. Recent cross-sectional studies found that balance control in older adults who bicycling regularly outdoors is better than matched controls. However, recommendation to bicycle outdoors for older adults may be dangerous. Consequently, there is a need to develop technologies that can improve balance reactive strategies for reducing falls in older adults.

Thus, there is a need in the art for improved systems and methods for training and enhancing balance control, to prevent falls of subjects in need thereof, in particular, disabled subjects and older adults, which are susceptible for loss of balance.

### SUMMARY

Aspects of the disclosure, according to some embodiments thereof, relate to a mechatronic perturbation stationary bicycle robotic system (also referred to herein as PerStBiRo), that provide unexpected perturbation during stationary cycling or pedaling. In addition, the PerStBiRo system provide implicit close-loop feedback control for the trainee’s trunk reactive balance responses that help to implement implicit learning to the trainee’s reactive balance control. In some embodiments, motion acquisition/capture unit is utilized to obtain information related to the training and performance of a user and/or provide real-time feedback on trunk and arms reactive balance responses during the cycling training session. According to some embodiments, further provided are related computer implemented methods of machine learning and artificial intelligence (AI) tools that are utilized to obtain data related to the training and balance performance of a user, analyze the related data and control/adjust the training session and/or provide training recommendations/training program(s).

According to some embodiments, the systems and methods disclosed herein are advantageous as they can specifically improve trunk, arms and lower limbs balance reactive responses in a safe, accurate and personalized manner. In some embodiments, the systems and methods can improve balance reactive responses in subjects such as older people, as well as pre-frail and frail older adults, Cerebral Palsy, Traumatic Brain Injury and partial spinal cord injured patients, and also in persons that cannot attend to perturbation programs that include treadmill walking.

According to some embodiments, there is thus provided a system that provides programmed and controlled small-to-moderate and unpredictable balance perturbations during stationary cycling to a user. In some embodiments, the system is further configured to provide one or more cognitive challenges to a trainee, during the balance perturbation training. In some embodiments, the system further includes a motion capture unit configured to capture/obtain/detect movement of the trainee during perturbations. In some embodiments, a processor unit of the system is configured, inter alia, to utilize data from the motion capturing unit and/or additional data related to the trainee’s performance (such as data related to the cognitive challenge performance) and based thereon to provide customized training program and/or adjust the training session.

According to some embodiments, there is provided a mechatronic bicycle simulator system for stimulating balance control of a subject, the system includes: a stationary training bicycle (STB); a moving platform; one or more motors; and a central control unit; wherein the moving platform is configured to provide external perturbation tilts to the STB, thereby stimulating balance of a subject situated on the STB.

According to some embodiments, the STB includes a pedal unit and a seat.

According to some embodiments, the relative position of the pedal unit and the seat is adjustable.

According to some embodiments, the pedal unit may be adjustable in height, resistance, force, tension and/or speed.

According to some embodiments, the seat may be adjustable in height and/or angle.

According to some embodiments, the system may include a harness configured to secure the subject (trainee) during the perturbations.

According to some embodiments, the moving platform may be mounted on an axis and may be configured to be in a fixed state or a floating state.

According to some embodiments, the system may be configured to provide intrinsic self-induced perturbations, when the moving platform is in a floating state. In some embodiments, the self-induced perturbations are during cycling.

According to some embodiments, the floating state of the moving platform is configured to be engaged during a time period between external perturbation tilts.

According to some embodiments, the system may further include a gear mechanism.

According to some embodiments, the gear mechanism may be configured to allow the transmission of motor rotation with a rotation axis of the moving platform by one or more ball bearings, thereby allowing the moving platform rotation and the balance external perturbation tilts.

According to some embodiments, the system may further include a motion control unit.

According to some embodiments, the system may further include a motion capture unit.

According to some embodiments, the motion capture unit comprises one or more video cameras, webcam camera, smartphone camera, and the like. In some embodiments, the video camera may be any type of suitable video camera, having any type of appropriate sensor, including, for example, CCD sensor, CMOS sensor, RGB sensor, and the like. In some embodiments, the cameras are depth video cameras.

According to some embodiments, the central control unit may be configured to control operation parameters of the motion control unit and/or the motion capture unit.

According to some embodiments, the system may further include a user interface and/or a display.

According to some embodiments, the external perturbation tilts are selected from: lateral perturbations (left and right tilt perturbations), Antero-posterior perturbations (forward and backward tilt perturbations), Vertical perturbations, Rotations around a vertical axis, or any combination thereof.

According to some embodiments, the lateral perturbations are in the range of about 0°-20° to each side and/or wherein velocity of the lateral perturbations is in the range of about 0-30 degree/sec and/or wherein acceleration or declaration is in the range of about 0-30 degree/sec².

According to some embodiments, the antero-posterior perturbations are in the range of about 0°-15° to each direction and/or wherein velocity of the antero-posterior perturbations is in the range of about 0-30 degree/sec and/or wherein acceleration or declaration is in the range of about 0-30 degree/sec².

According to some embodiments, the frequency of the lateral perturbations and/or the antero-posterior perturbations is in the range of about 1-15 times per minute.

According to some embodiments, the vertical perturbations are in the range of about 0-15 cm in each direction, and/or wherein velocity of the vertical perturbations is in the range of about 0-40 cm/sec and/or wherein acceleration or declaration is in the range of about 0-40 cm/sec² and/or wherein the frequency is in the range of about 1-10 perturbations/min.

According to some embodiments, the rotation perturbations are in the range of about 0-10 cm in each direction, and/or wherein velocity of the rotation perturbations is in the range of about 0-40 cm/sec and/or wherein acceleration or declaration is in the range of about 0-40 cm/sec² and/or wherein the frequency is in the range of about 1-10 perturbations/min.

According to some embodiments, the central control unit is further configured to provide cognitive challenge to the trainee, during at least a portion of a perturbation system.

According to some embodiments, the cognitive challenge may include cognitive games and/or cognitive tasks. In some embodiments, the cognitive challenge may be provided in various levels of difficulty or complexity.

According to some embodiments, the central control unit may include a processing unit configured to execute a computer program configured to determine performance of the subject during perturbations and/or determine further perturbations training sessions (training plan).

According to some embodiments, the in the computer program may include machine learning algorithms.

According to some embodiments, the determination of the performance of the subject during perturbations is performed in real time, based at least in part on data related to balance reactive response performance of the subject during perturbations and optionally, during the cognitive tasks.

According to some embodiments, the determination of further perturbations training sessions (training plan) is based at least in part on data related to the performance of the subject during a previous perturbation session.

According to some embodiments, the motion control unit is configured to direct the motor operation, based on the training plan.

According to some embodiments, the external perturbations are provided in a triangular motion profile (acceleration-deceleration) for each perturbation.

According to some embodiments, operating parameters of a training session may include: type of perturbation, maximum acceleration/deceleration of a perturbation, maximum velocity of a perturbation, magnitude of a perturbation, angle of perturbation, number of perturbation repetitions, the delay time between the perturbations, relative position between the seat and the pedal unit, operating parameters of the pedal unit, or any combination thereof.

According to some embodiments, the processing unit is configured to provide real-time feedback to the subject regarding reactive balance reaction following a perturbation or perturbation session.

According to some embodiments, one or more of the provided external perturbations are unexpected.

According to some embodiments, the central control unit is configured to allow a trainer to determine, select or confirm a training plan and/or control one or more balance exercise parameters.

According to some embodiments, the system may further include adjustable gripping handles.

According to some embodiments, the gripping handles comprises a heart rate sensor and/or a pressure sensor.

According to some embodiments, the training system further includes a communication unit configured to allow wired and/or wireless communication.

According to some embodiments, there is provided a method for training or improving balance control of a subject, the method includes one or more of the steps of:
providing one or more unexpected external perturbations to a subject using the training system disclosed herein,
detecting a reactive balance response of the subject to the perturbations based on data acquired by the motion capture unit of the system;
analyzing the detected reactive and proactive balance response; and
providing a feedback to the subject if the balance response is determined to be above a threshold.

According to some embodiments, the method may further include providing a cognitive challenge to the subject and determine a cognitive performance of the subject, based on the response to the cognitive challenge.

According to some embodiments, the cognitive challenge may be provided in synchronization with an unexpected external perturbation.

According to some embodiments, the reactive balance response threshold is customized to the subject.

According to some embodiments, the reactive balance response threshold may be determined based on calibration and/or based on previous training sessions.

According to some embodiments, the feedback may include stopping the perturbation and returning the moving platform to a neutral (i.e., vertical) position.

According to some embodiments, the analysis of the detected balance response and/or cognitive performance may be performed by a computer program comprising AI algorithms.

According to some embodiments, the computer program may be configured to provide feedback to the user indicative of the performance of the subject in the reactive balance response and/or the cognitive stimulation.

According to some embodiments, the computer program is further configured to adjust operating parameters of the training session based at least in part on the analyzed reactive balance response.

According to some embodiments, the computer program may be further configured to determine or recommend operating parameters of a following training session and/or a training plan, comprising two or more consecutive training sessions.

According to some embodiments, there is provided a computer-readable storage medium having stored therein machine learning software, executable by one or more processors for executing the training method as disclosed herein.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1 a pictogram of a mechatronic bicycle simulator system, according to some embodiments. Shown in FIG. 1 is the system while being used by a subject (trainee), under the supervision of a trainer;

FIG. 2 a pictogram of a gear mechanism of a mechatronic bicycle simulator system, according to some embodiments;

FIGS. 3A-3D, show schematic illustrations of various types of external perturbations in an exemplary training system, according to some embodiments. FIG. 3A- lateral perturbations; FIG. 3B- Antero-posterior perturbations; FIG. 3C- vertical perturbations; FIG. 3D- rotation perturbations;

FIGS. 4A-C, show schematic illustrations of various relative positions of the seat and pedal unit in exemplary training system, according to some embodiments. FIG. 4A -Kayak position; FIG. 4B- Bicycle position; FIG. 4C- pedaling—standing position;

FIG. 5, show schematic illustration of a standing position for assessment of balance performance assessment, according to some embodiments;

FIG. 6 is a flowchart of the communication routes between various system components, according to some embodiments. The arrows represent the propagation and directions of information and data between each component and what it is connected to. The light boxes represent the main system parts that also receive or transmit communications, while the darker gray boxes represent Intermediate components that help with connection or communication;

FIG. 7 shows an exemplary history tab of a user interface, displaying training data of a training session, according to some embodiments;

FIG. 8, a flow chart of steps of AI algorithms utilized in balance training system, according to some embodiments;

FIG. 9 shows a table detailing the PerTSBR intervention training program of Example 2. Listed are the intensity and the progression levels of the training sessions. Details of the external perturbations, cognitive tasks, and bicycle resistance during the 22 potential training sessions. Abbreviations: deg = degrees, sec = seconds, sec² = second * second, Vel. = velocity, Acc. = acceleration;

FIGS. 10A-C: show a sample of upper body movements analysis of a subject on a training system. A sample of upper body movements (horizontal lines) and the PerStBiRo system’s stationary bicycle (horizontal black lines) by time during a calibration phase (left of the dotted line in FIG. 10B and FIG. 10C) and during balance perturbation exercise phase (FIG. 10A), focusing on the upper body reactive balance response of an 82-year-old trainee following a programmed 20° right tilting perturbation (FIG. 10A and gray timelines in FIGS. 10B and 10C). Shoulder line angle—the angle of the participant’s shoulder line and the ground (FIG. 10B, horizontal purple line); Head-Neck angle—the angle of the participant’s head-to-neck line and the vertical line to the ground (FIG. 10B, horizontal green line). Points 1 + 2 indicate external perturbations that lead to sharp and large upper body balance reactions. The time range 1-2 indicates internal perturbations, the upper body oscillations when riding on an unstable surface as seen the horizontal black lines, that represent the stationary bicycle angles, are not exactly on the vertical 0° position;

FIGS. 11A-B show example of the ability of the training system to monitor and identify skill acquisition of hands-free pedaling during training session of an 86 year old traineed. FIG. 11A -a sample of about 20 seconds of a first training session. The 86 years old trainee released his hands from the handlebars (about 94 seconds of the training session), which was immediately accompanied with upper body instability, i.e., shoulder and head angle instability, during pedaling [purple (α1) and green (α2) line]; FIG. 11B - the end of the first training session (1,100-1,120 seconds of the training session). A sample that represents better upper body stability (i.e., lower amplitudes) during pedaling [purple (α1) and green (α2) lines]; and

FIGS. 12A-C - show example of the ability of an 86-years old trainee to reactively respond to unannounced perturbations during hands-free pedaling along the training sessions. FIG. 12A- Low-magnitude perturbations in block right-left training of 2.5° tilt (i.e., the black arrows). A sample of 26 seconds that represents the participant’s ability to consistently react to perturbations [shoulder line angles (purple line) react in the opposite direction and related to the black line perturbations]; FIG. 12B- An example of effective trainee’s reactive balance responses during random unexpected moderate-magnitude (6°-10° tilt) perturbation training. A sample of 30 seconds that represents generally organized and controlled shoulders/trunk movements [shoulder line angles (purple line] during hands-free pedaling, and particularly organized and effective upper-body balance responses (shoulders’ response, represented by purple line rises in a manner adapted to the perturbations); FIG. 12C- A sample of 60 seconds during the fourteenth session that represents the participant’s ability to respond reactively to high-magnitude (8°-12° tilt) random unannounced external perturbations (spikes in the black line that represent the stationary bicycle training angles, black arrows) and respond proactively to self-induced perturbations during the “floating” mode of the moving platform (gentle long humps in stationary-bicycle-training black line, red arrows). Generally, the shoulders’ balance responses show an organized response appropriate to the challenge of balance, i.e., the shoulders’ purple line move in the opposite direction when the self-induced perturbations occurred and usually also in the case of unannounced external perturbations.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

As used herein, the terms “bicycling”, “cycling” and “pedaling” may interchangeably be used. The terms relate to the operation (such as turning or pressing) of corresponding pedal(s) by a user. In some embodiments the terms relate to stationary cycling.

As used herein, the term “bicycle” relates to a stationary cycling apparatus, which includes turnable pedals. In some exemplary embodiments, the cycling apparatus may be in the form of stationary bicycle, stationary unicycle, a seat and associated pedal(s), and the like.

As used herein, terms “subject” and “trainee” may interchangeably be used. The terms relate to a user which is training on the simulator system.

According to some embodiments, there is provided a mechatronic bicycle simulator system (also referred to herein as Perturbation Stationary Bicycle Robotic system (PerStBiRo)) that provides various types of expected and unexpected perturbations which challenge the proactive and reactive balance control in subjects, such as, older adults, during bicycling, in safe sitting position, suitable for subjects (such as older adults) at different levels of functioning.

According to some embodiments, the system disclosed herein includes a mechatronic device that provides unexpected balance perturbations during (stationary) bicycling in safe and secure environment.

In some embodiments, as detailed below herein, the perturbations may include lateral perturbations (right and/or left tilt), Antero-posterior perturbations (forward and/or backward tilt), Vertical perturbation (upward and/or downward perturbation), rotation around a vertical axis, or combinations thereof. Each possibility is a separate embodiment.

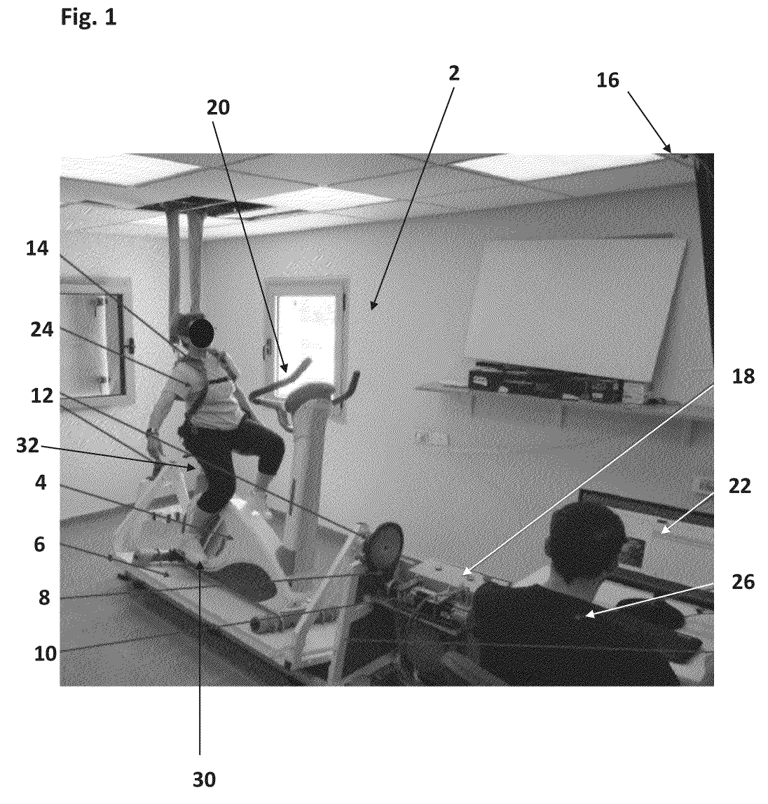

Reference is made to FIG. 1, which shows a pictogram of a mechatronic bicycle simulator system, according to some embodiments. Shown in FIG. 1 is system 2, while being used by a subject (trainee 24), which in this example is trained under the supervision of a trainer (26). As shown in FIG. 1, System 2 includes stationary training bicycle 4, which are situated/mounted on/attached to a moving platform 6. Moving platform 6 is configured to provide various types of external perturbations by being moved/tilted using gears and gear mechanisms 8 and ball bearings 12, which are powered by motor 10 (that may be any type of suitable motor, such as, servo motor) and controlled by motion control unit 18. In some embodiments, the gear ratios combine the servo-motor rotation with the platform rotation axis by two ball bearings, one on each side of the platform, allowing the platform rotation and hence the balance perturbation tilts. According to some embodiments, the moving platform is an open iron frame, for example, 152-cm long and 64-cm wide, located within another stationary iron frame that are interconnected by two ball bearings allowing tilt motion in lateral directions. The stationary training bicycle (STB) is mounted on the moving platform and secured by four metallic arcs that bind its legs to the moving platform. the moving-platform frame is connected from both ends (back and front) to two ball bearings. The front ball bearing connects to a gear mechanism and the servo motor. In some embodiments, as detailed below herein, the moving platform may be placed on an axis and transition between two states: a “fixed state”, under which the moving platform can provide external perturbations (i.e., the movement thereof is controlled by the motion control system via the gears mechanism) , or a “non-fixed” or “floating” state, under which the platform is non-stable (i.e., it is not stationary/fixed), which provides “intrinsic” perturbation, by forcing the trainee to balance him/herself on the platform. As further shown in FIG. 1, system 2 includes a motion capture unit 16, which includes one or more motion capturing devices (such as video cameras), which is configured to detect motion of the subject, in particular, during perturbation. In some exemplary embodiments, the motion capture camera may be mounted at a 45° horizon angle at a height of about 2.8 m and 3 m in front of the subject’s sitting position for the best motion capture of the trunk and upper body reactions, without being hidden from the STB’s handlebars. As shown in FIG. 1, the system further includes safety harness and/or straps, which are configured to secure the subject and prevent or hold the subject, so he does not accidently fall during a training session. Subjects may be outfitted with a harness attached to an overhead rail. The harness was adjusted so that, with full body weight support by the harness, the subject’s knees could come close to, but not touch, the platform. In some embodiments, the instrumented harness is configured to objectively evaluate/measure harness support which may also provide information for the trainer for cases in which harness support is stretched. In some embodiments, a cutoff level of harness support (for example, about 20% body weight) which is indicative that the trainee failed to respond appropriately to the provided balance stimulation. The system further includes a control unit 22, which may include one or more displays, and/or processors, which are configured to control operation of the system, obtain data from the motion capture unit, analyze data related thereto, provide real time feedback to the subject and/or trainer, asses subject performance, determine or suggest a training session or training program, provide, present and/or assess cognitive tasks to subject, and the like, or combinations thereof. As further shown in FIG. 1, system 2 includes gripping handles (handlebar) 20, which may be used to assist the subject in sitting or resting. In some embodiments, the gripping handles are not used during a training session. As further shown in FIG. 1, STB 4 includes a pedal unit 30, having pedals configured to be turned/rotated during training, and a seat 32, on which the subject is placed during training. In some embodiments, as further detailed below, the relative position of the seat and the pedal unit is adjustable. In some embodiments, the height and/or angle of the seat are adjustable.

According to some exemplary embodiments, the system may weight about 90 kg and its hardware includes one or more of the following components: moving-platform frame, stationary frame, STB, servo motor, motion control system, gears and gear mechanism, two ball bearings and motion capture unit, as illustrated in FIG. 1. The details of exemplary components, are listed in Table 1 in Example 1, below herein.

According to some embodiments, the STB may be mounted on, attached to, fixed to or be associated with the moving platform., in a reversible or non-reversible manner.

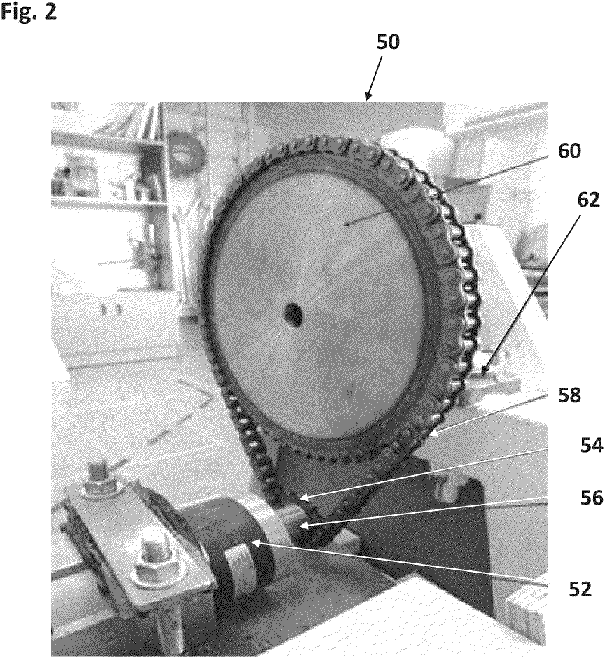

Reference is now made to FIG. 2, which shows a pictogram of a close up view of a gear mechanism of a mechatronic bicycle simulator system, according to some embodiments. As shown in FIG. 2, gear mechanism 50 includes motor 52, and a set of gears and transmissions, configured to move the moving platform and thereby provide perturbations to the subject. As shown in FIG. 2, gear mechanism 50 includes small external gear 54, cylinder 56, motor chain and internal transmission system 58 and large external gear 60. Also shown is front ball bearing 62 (shown as ball bearing 12 in FIG. 1). In some embodiments, the moving platform is connected to the series of gears and the servo motor having a power rating in the range of about of 2.5-4 kw (such as, for example, 2.97-3.38 kw). In some embodiments, the gear ratios combine the servo-motor rotation with the platform rotation axis by two ball bearings, one on each side of the platform, allowing the platform rotation and hence the balance perturbation tilts. In the gear mechanism 50 itself which is illustrated in FIG. 2 there are two external gears (large (60) and small (54)) that connects by a motor chain (58) and therefore there are two internal transmissions with a gear ratio of approximately 1:5 (for example, in the range of 1:10). The servo motor (52) is connected to a cylindrical component (56) which may be welded to the small external gear 54. In some embodiments, the motor may have a speed of 1000-5000 rpm (such as, maximum speed of 3000 RPM), and peak torque in the range of 5-15 Nm (such as, 10.8 Nm).

According to some exemplary embodiments, the system may provide maximum right and left perturbation tilt angle (each side) in the range of about 5-30° (such as, for example, of 20°) with acceleration and deceleration in the range of 20-40 m/s^2 and maximum velocity in the range of about 20-40 m/s (for example, maximum acceleration and deceleration of 30 m/s^2 and maximum velocity of 30 m/s). The motor may be controlled by a motion control system and optionally a motion capture unit (which may include any type of video camera, such as, for example, a Microsoft Kinect system, Intel RealSense™ depth camera, webcams, smartphone cameras, and the like), both may be controlled by a main processing unit executing a set of instructions (i.e., a computer program). In some embodiments, the computer program may be executed on a host PC that may also serve as a user interface. By the computer program command, the motion control system can direct the motor rotation based on a programmed training plan, which is usually that of a triangular motion profile for each perturbation (acceleration-deceleration). The computer program may allow the trainee or a trainer to determine the training plan and control one or more of the balance exercise parameters, such as, for example, but not limited to: maximum acceleration/deceleration, maximum velocity, angle of perturbation, number of right/left perturbation repetitions, the delay time between the perturbations, and the like, or any combination thereof. In some embodiments, the computer program may also allow controlling a motion capture system/unit (such as, a video camera, high definition video camera, depth video camera, and the like) that can provide a real-time feedback as to the trainee’s balance reaction following a perturbation.

In some embodiments, once unexpected balance perturbation is given, when an appropriate reactive balance reaction is detected by the motion capture unit, configurable by the computer program, or by the trainer (also referred to as health care provider, therapist) in, the moving platform rotation (the perturbation) is stopped, and the motor returns the system to its vertical position (its neutral/zero position) by motor counter-rotation. In some embodiments, the user (also referred to as subject/trainee) may be harnessed to safety straps, such as, safety straps attached to the ceiling, or to an arm, as detailed below. In addition, the program may be configured to save/store a file that logs the exercise(s) performed, for post-training analysis.

According to some embodiments, the PerStBiRo system may be used to implement Motor learning of balance. Motor learning refers to the human internal processes associated with practice a particular movement that leads to relatively permanent changes in the capability for responding. Motor learning process improves with practice many repetitions of motor performance, leading to improvement in the person’s capability in producing the desired action. Varied practice in random order results in better motor learning. In some embodiments, during a customized training program, the desired result is for the trainee to perform an effective reactive balance reaction with the trunk, upper body and arms, as well as leg musculature to recover their balance from a perturbation during bicycling.

In some embodiments, the PerStBiRo system, utilizing its computer program and motion control units, may be used to expose the trainee to repeated random unexpected balance perturbations by tilting the moving platform (the STB and the trainee) repeatedly to varied certain programmed tilt angles, so the patient may learn better how to recover their balance more efficiently along a training session and at whole training course. For even more increasing the trainee’s motor learning process of acquiring effective reactive balance reactions, the motion capture system (monitors the patient’s whole-body joints, and following a perturbation, detects the trainee’s reactive balance reactions and determines whether the response was effectively enough. When an effective balance reaction is performed, the motor control system stops automatically the perturbation and returns immediately the system to its neutral/vertical position (0°). This task intrinsic feedback, the immediate real-time balance response feedback, provides the learner (trainee) an implicit cue to successful reactive balance response and gives best possible motor learning implementation.

According to some embodiments, the PerStBiRo system may provide multidirectional external machine-induced programmed unannounced (unexpected) perturbations. According to some embodiments, the perturbations may be of various types and directions and may include any combination. According to some embodiments, the external perturbations may be selected from: lateral perturbations (right and left tilt perturbation), antero-posterior perturbations (backwards and forwards tilt perturbations), vertical perturbations (up and down), perturbations that are rotation around a vertical axis, or any combination thereof. In some embodiments, the perturbations may be provided in a triangular motion profile (acceleration-deceleration). In some embodiments, each training session may include one or more types of perturbations, at any desired order, at any desired length, at any desired time interval and/or at any desired complexity.

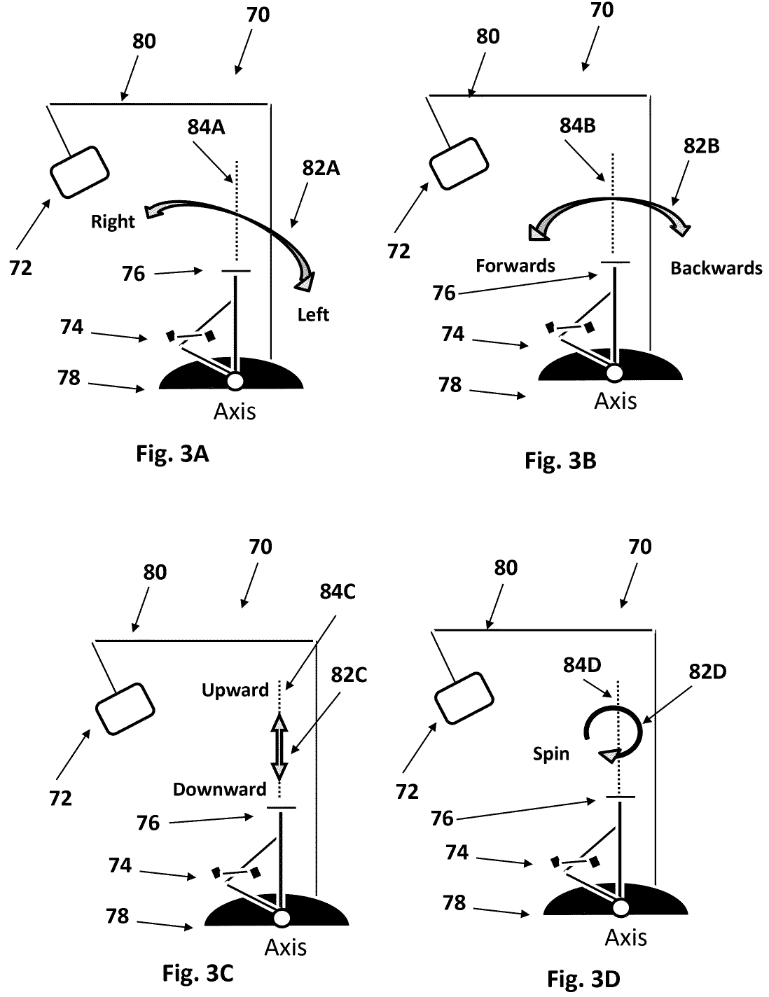

Reference is now made to FIGS. 3A-3D, which show schematic illustrations of various external perturbations in an exemplary training system, according to some embodiments. As shown in FIG. 3A, exemplary system 70 includes at least seat 76, pedal unit 74 and moving platform 78, on which the pedal unit and seat (i.e., STB) are mounted/attached. Further shown is stationary arm 80 which is configured to hold the motion capture unit 72 (as well as display, user interface, processing unit, and the like). Illustrated in FIG. 3A are lateral (left-right) perturbations 82A (relative to axis 84A). According to some embodiments, the magnitude of the perturbations may be in the range of about 0°-30° (for example, 0-20 °) to each side. In some embodiments, the velocity of the movement may be in the range of about 0-30 degree/sec, or any subranges thereof. In some embodiments, the acceleration of the movement may be in the range of about 0-30 degree/sec², or any subranges thereof. In some embodiments, the deceleration of the movement may be in the range of about 0-30 degree/sec², or any subranges thereof. In some embodiments, the frequency of the perturbations may be in the range of about 1-20 (such as, 1-15) perturbation/minute, or any subranges thereof.

Reference is made to FIG. 3B which illustrates antero-posterior (left-right) perturbations 82B (relative to axis 84B) in exemplary system 70. According to some embodiments, the magnitude of the perturbations may be in the range of about 0°-30° (for example, 0-20°) to each side. In some embodiments, the velocity of the movement may be in the range of about 0-30 degree/sec, or any subranges thereof. In some embodiments, the acceleration of the movement may be in the range of about 0-30 degree/sec², or any subranges thereof. In some embodiments, the deceleration of the movement may be in the range of about 0-30 degree/sec², or any subranges thereof. In some embodiments, the frequency of the perturbations may be in the range of about 1-20 (such as, 1-15) perturbation/minute, or any subranges thereof.

Reference is made to FIG. 3C which illustrates vertical (up-down) perturbations 82C in exemplary system 70. According to some embodiments, the magnitude of the perturbations may be in the range of about 0-30 cm (such as, for example, 0-15 cm) in each direction, or any subranges thereof. According to some embodiments, the velocity of the perturbations may be in the range of about 0-40 cm/sec, or any subranges thereof. In some embodiments, the acceleration of the perturbations may be in the range of about 0-40 cm/sec², or any subranges thereof. In some embodiments, the deceleration of the perturbation may be in the range of about 0-40 cm/sec², or any subranges thereof. In some embodiments, the frequency of the perturbations may be in the range of about 1-10 perturbation/minute, or any subranges thereof.

Reference is made to FIG. 3D which illustrates rotation perturbations 82D (around vertical axis 84D) in exemplary system 70. According to some embodiments, the magnitude of the perturbations may be in the range of about 0-10 cm above and below the zero/vertical position of the seat at the non-perturbed condition. According to some embodiments, the velocity of the perturbations may be in the range of about 0-40 cm/sec, or any subranges thereof. In some embodiments, the acceleration of the perturbations may be in the range of about 0-40 cm/sec², or any subranges thereof. In some embodiments, the deceleration of the perturbation may be in the range of about 0-40 cm/sec², or any subranges thereof. In some embodiments, the frequency of the perturbations may be in the range of about 1-10 perturbation/minute, or any subranges thereof.

According to some embodiments, in addition to the external perturbations provided by the motors, internal self-induced perturbations may also be provided by the system. According to some embodiments, perturbations may be provided in two forms, “internal” and external balance perturbations. As detailed above, the system is configured to provide external machine-induced programmed unannounced lateral perturbations. Additionally, the system may be configured to provide internal self-induced perturbations, for example, during pedaling on an unstable “floating” movable platform. In some embodiments, the moving platform may transition between two states/modes: fixed state (whereby it is configured to be moved by the motion control unit and provide external perturbations) and floating (non-fixed) state, in which the platform may be slightly unstable, allowing self-induced tilting. According to some embodiments, in the time interval between two consecutive external perturbations, the moving platform may be in a fixed mode/state or in the “floating” mode/state, where it can similarly to a surfboard floating on the water and is subjected to the forces exerted on it by the subject during pedaling. According to some embodiments, this unstable mode/state may be programmed by the user for the time interval between the external perturbations. According to some embodiments, the internal self-induced perturbations may thus be provided by the unfixed (unstable, floating mode) of the moving platform of the system. According to some embodiments, such self-induced perturbations mimic or simulate outdoor bicycling, and can be part of a proactive balance control training. According to some embodiments, such internal perturbations may be included for advanced trainee subjects. According to some embodiments, during the unfixed “floating” mode the motor may be released (i.e., at least a portion of the gear mechanism not engaged with the moving platform), for example, in the time interval between the external perturbations.

According to some embodiments, when calibrating the system, a customized calibration phase may be performed in the same fixed or unfixed floating state, as the one expected to be utilized during the training. In some embodiments, the fixed mode/state is when the system is locked/fixed vertically and used as a regular stationary bicycle unit. In some embodiments, the “floating” mode is when the moving platform is unfixed and unstable, floating like a surfboard and is subjected to the forces acting upon it by the pedaling subject.

According to some embodiments, the pedaling unit of the system includes a set of pedals that may be rotated by the subject and/or the system. According to some embodiments, the pedaling unit may be configured to monitor various pedaling related parameters, such as, for example, but not limited to: pedaling time, pedaling distance, pedaling resistance, determine pedaling intensity based on distance, heart rate, and pedaling load, and the like, or any combinations thereof. Each possibility is a separate embodiment. In some embodiments, the pedaling unit may be configured to monitor various pedaling related parameters during perturbation training and/or cognitive training. In some embodiments, the speed, resistance, position and/or height of the pedaling unit may be adjustable automatically or manually. In some embodiments, the height of the pedal unit may be in the range of about 2-60 cm above the ground (floor) or the moving platform. In some embodiments, the pedal unit may be positioned in a forward position relative to the seat, wherein the pedal unit may be positioned about 5-150 cm forward relative to the seat.

According to some embodiments, the position of the seat and the pedal unit may be adjustable. In some embodiments, the relative position of the seat and the pedal unit may be adjustable. According to some embodiments, the balance perturbation training can be performed in various body positions according to increasing degrees of difficulty, with or without pedaling. In some embodiments, the moving seat and/or the pedal unit can be adjusted. Accordingly, the seat and/or pedal unit, can suit/fit subjects of various sized and/or subjects having different ranges of motion in the thighs and knees. In some embodiments, the seat can be adjusted up and down in the range of about 20-90 cm above the floor or moving platform. In some embodiments, the pedal unit can be adjusted up and down in the range of about 5-50 cm above the floor or moving platform. In some embodiments, the pedal unit may further be adjusted forward and backward in the range of about 20-120 cm related to the vertical position of the moving seat. According to some embodiments, the seating position can be customized according to the comfort of the user and according to the level of difficulty of the training.

According to some exemplary embodiments, sitting or training positions may include, for example, but not limited to: “Kayak position” (which is of easy difficulty level), “bicycle position” (which is of moderate difficulty level) and “Pedaling-Standing position” (which is high degree of difficulty), and the like, or combinations thereof.

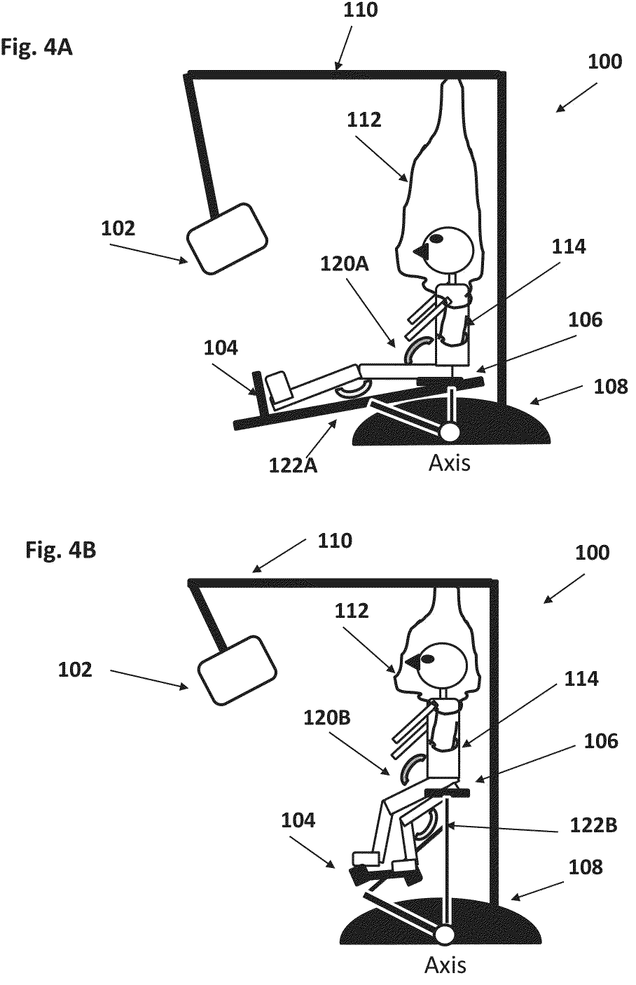

Reference is now made to FIGS. 4A-C, which schematically illustrates various relative positions of the seat and pedal unit in exemplary training system, according to some embodiments. As shown in FIG. 4A, exemplary system 100 includes at least seat 106, pedal unit 104 and moving platform 108, on which the pedal unit and seat are mounted/attached. Further shown is stationary arm 110 which is configured to hold the motion capture unit 102 (as well as display, user interface, processing unit, and the like). Further shown are harness 112, which are secured/connected/attached to trunk 110, and are configured to secure the subject using the system (shown as exemplary subject 114). In system 100, the seat-pedal unit are positioned in exemplary “kayak position”. In such a setting, angle 120A (body-thigh angle) may be in the range of about 90°-110°. Angle 122A (Thigh-knee angle) may be in the range of about 170-90°. In some embodiments, in such a setting, the seat height can be adjusted to be about 20-50 cm above the floor or the moving platform. In some embodiments, in such a setting, the pedal unit may be adjusted to 5-20 cm above the floor or moving platform and may be placed about 70-120 cm froward relative to the seat. In some embodiments, the kayak position mimics sitting position in a kayak. the sitting position is close to the floor, with Body-Thigh angles of about 90-110 degrees and a Thigh-Knee angles in the range of about 170°-90°. In some embodiments, in this position the pedal unit serves as a place to rest the feet.

Reference is made to FIG. 4B, in which in system 100, the seat-pedal unit are positioned in exemplary “bicycle position”. In such a setting, angle 120B (body-thigh angle) may be in the range of about 110° -145°. Angle 122B (Thigh-knee angle) may be in the range of about 170-60°. In some embodiments, in such a setting, the seat height can be adjusted to be about 45-90 cm above the floor or the moving platform. In some embodiments, in such a setting, the pedal unit may be adjusted to about 15-50 cm above the floor or moving platform and may be placed about 20-60 cm froward relative to the seat. In some embodiments, the bicycle position mimics various standard sitting positions, such as sitting on a standard bicycle, such that when perturbation is executed, the seat moves together with the pedal unit.

Reference is made to FIG. 4C, in which in system 100, the seat-pedal unit are positioned in exemplary “pedaling-standing position”. In such a setting, angle 120C (body-thigh angle) may be in the range of about 145°-180°. Angle 122C (Thigh-knee angle) may be in the range of about 180-70°. In some embodiments, in such a setting, the seat height can be rotated and serve as rear backseat. In some embodiments, in such a setting, the pedal unit may be adjusted to about 5-50 cm above the floor or moving platform and may be placed about 20-60 cm froward relative to the seat. In some embodiments, the Pedaling-Standing position mimics standing on the pedals while pedaling.

According to some embodiments, the system may include gripping handles. In some embodiments, the gripping handles may be detachable and adjustable according to, for example, the height of the seat, and may be located in front or on the side of the moving seat. According to some embodiments, grip handles may be used for the trainee’s positioning, and also be used by those who afraid of hands-free pedaling (for example, at the beginning of the training session) or whose initial balance ability level is too low for a hands-free pedaling. In some embodiments, this level of training represents little actual challenge to the postural control system. The goal of the training at this level is mainly directed towards a cognitive understanding of the exercises and an improvement of self-confidence for trainees. Nevertheless, the trainee is required/asked to perform the training with no or minimal external support (i.e. without holding the handles). According to some embodiments, the grip handles may further be used for heart rate monitoring. According to some embodiments, pressure sensors may be located on the grip handles. The pressure sensors may be configured to monitor the hands use for holding the handles during training, which can be used as a measure of success in training (for example, hands-free pedaling of 80% of the training time may be required to advance to the next level). In some embodiments, the pressure sensors may further be used for creating training session which include combined training intervals with and without hands gripping.

According to some embodiments, the system may include a communication unit. The communication unit may be configured for wired communication or wireless communication (using, for example, a cellular network, Wi-Fi and/or Bluetooth). According to some embodiments, the communication unit may allow communication with one or more servers, remote server(s), remote control station, other training units, and the like. In some embodiments, the communication unit allow the transfer or sharing of information, for example to and from servers, to and from other stimulation systems, to and from remote trainers’ units, to and from remote central control units, and the like. In some embodiments, the communication unit is functionally and/or physically associated with the control unit.

According to some embodiments, the system may further allow or be utilized to provide training assessment. According to some embodiments, the assessment sessions may be used to enable the user/trainee to follow and monitor his/her reactive balance, functional balance and/or cognitive progression after the balance perturbation training program (optionally combined with cognitive tasks, as detailed herein).

According to some embodiments, the trainee’s balance performance assessment can be performed in various forms, including, for example, by a reactive balance examination on the dedicated moving platform (i.e., the ability to control the center of mass motion is assessed - lower center-of-mass movement is indicative of better reactive balance ability); or by rotating the motion capture unit into the room space and performing a functional balance examination in the home environment (including, for example, sit to stand task, walking task while gait analysis is assessed, single leg stance time). In some embodiments, such assessment options may last about 2-30 minutes (for example, 15 minutes), and the subject is instructed to follow the instructions presented on the display by the system control unit. In some embodiments, the score of the assessment session may be presented on the display (screen) and optionally stored in a memory. In some embodiments, the reactive balance score and/or functional balance score may be used by the computer program of the system (in particular, utilizing artificial intelligence algorithms, as detailed herein), to adjust future training sessions. In some embodiments, a cognitive assessment may also be performed a sitting position in front of the display. In some exemplary embodiments, the user may execute the cognitive tasks displayed on the screen. In some exemplary embodiments, the cognitive tasks may include various types of tasks, such as, for example, but not limited to: the differences between two pictures, find the odds one out, identify a world location, perform colored Stroop test, and the like, or any combination thereof. In some embodiments, such session may last for about 5-60 minutes (for example, about 15 minutes). In some embodiments, the number of correct answers, memory capability and response time for answering may be assessed and used to provide a cognitive score for a specific assessment session.

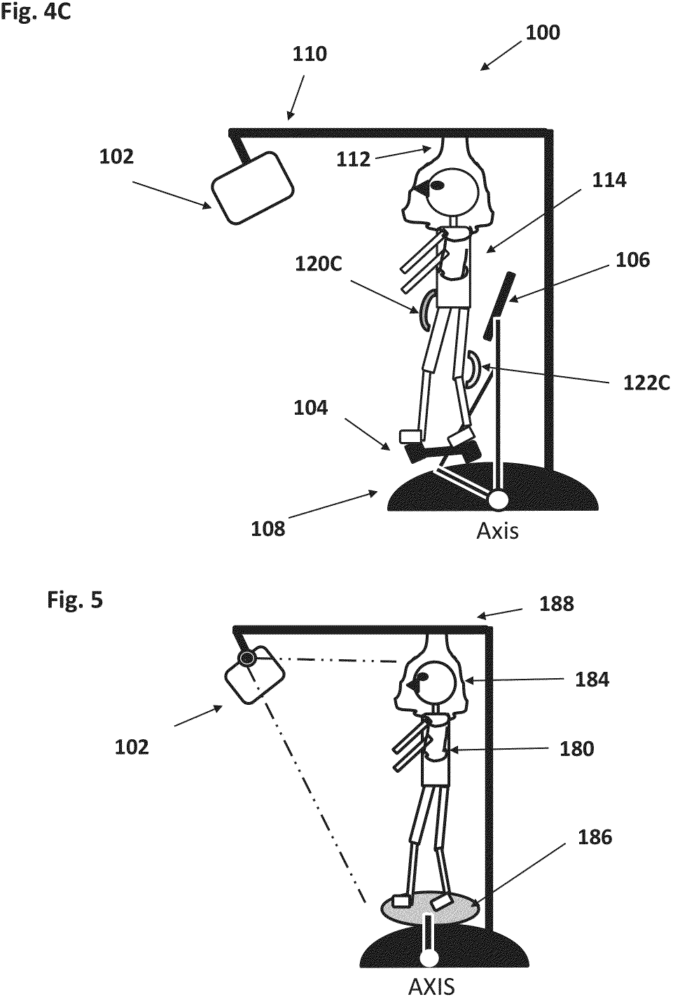

Reference is now made to FIG. 5, which schematically illustrates a standing position for assessment of balance performance assessment, according to some embodiments. The standing position option is for advanced trainee or for assessment sessions, when the trainee can monitor his/her reactive balance progression in specific assessment sessions. As shown in FIG. 5, subject 200 may stand on a standing platform 186. The subject is secured by harness 184 to trunk 188. Further shown is motion capture unit 182. To get to the standing option, the pedaling unit may be removed and instated, a standing platform is attached/placed/installed on the axis. The standing position allows for perturbations in different directions. Because it is a very high level of balance training, and for safety reasons, the adjusted parameters of each type of perturbation are reduced. In some embodiments, the external perturbations may include lateral perturbations (right and left tilts), antero-posterior perturbation, vertical perturbations and/or rotation around an axis perturbation. In some embodiments, perturbation parameters of lateral perturbations may be in the magnitudes of about 0°-10° to each side; velocity of about 0-20 degree/sec; acceleration of about 0-20 degree/sec²; deceleration of about 0-20 degree/sec²; and frequency of about 1-6 perturbation/min. In some embodiments, antero-posterior perturbation parameters may include: magnitudes of about 0°-10° in each direction; velocity of about 0-20 degree/sec; acceleration of about 0-20 degree/sec²; deceleration of about 0-20 degree/sec²; Frequency of about 1-6 perturbation/min. In some embodiments, vertical perturbation parameters (up and down) may include: magnitudes in the range of about 0-5 cm in each direction; velocity of about 0-30 cm/sec; acceleration of about 0-25 cm/sec²; deceleration of about 0-25 cm/sec²; frequency of about 1-6 perturbation/min. In some embodiments, rotations around a vertical axis parameter may include: magnitudes in the range of about 0-15 degrees each direction; velocity of about 0-20 cm/sec; acceleration of about 0-20 cm/sec²; deceleration of about 0-20 cm/sec²; Frequency of about 1-6 perturbation/min.

According to some embodiments, the system if further configured to provide various types of cognitive challenges to a user, to be performed along with the perturbations. According to some embodiments, the cognitive concurrent tasks may be displayed on the user screen and may be provided by the processing unit during balance perturbation training while hands-free pedaling in sitting (or in some cases standing positions). In some embodiments, concurrent cognitive visual tasks may be provided/included to distract the trainee, thus, facilitating implicit learning and automatization of reactive balance responses, similar to everyday situations where balance is lost unexpectedly. According to some embodiments, the level and/or type of the cognitive task may be adjusted according to the user physical and/or cognitive ability, thereby allowing a customized training program.

According to some embodiments, various types of cognitive challenges (cognitive stimulations) may be provided, including, for example:
A) cognitive task(s) that are not related to the physical balance tasks. Such tasks may include, for example, find the differences between two pictures, find the odd one out, recognize famous places in the world, colored Stroop test and the like, or any combination thereof. This type of tasks may be provided for beginner trainee or for those whose balance skills are low. In these task the correct and incorrect answers as well as time to answer may be determined and stored/recorded. When the user succeeds in X% (such as, for example, 80%) of the tasks and his/her average time to answer is lower than Y seconds (for example, about 10-20 seconds), the next difficulty level may be suggested or implemented for the next training session. In some embodiments, the next level may include cognitive task(s) that are not related to the physical balance tasks but reaction time for responding is measured and recorded. Such exemplary tasks include, for example, tasks find the next number in invoice series, find a synonym phrase for a presented word and the like. These tasks can also be provided for beginner trainee that has moderate balance skills. When the user succeeds in X% (such as, for example, 80%) of the tasks and his/her average time to answer is lower than Y seconds (for example, about 3-10 seconds), the next difficulty level may be suggested or implemented for the next training session.
B) balance gaming - another type of cognitive tasks includes tasks that are related to the physical balance tasks. Such tasks may be in the form of games, that may depend on the siting position during the balance training and designed for users with moderate balance skills and above. The balance games train, in various level, fast balance responses, memory capability, information processing, motor planning, and the like, or any combination thereof. Each possibility is a separate embodiment. In some embodiments, real time feedback may be provided for balance performance. In some embodiments, the games include such games as avoiding obstacles or obstacles popping up along the way while cycling or kayaking. In some embodiments, while in standing position the games may include avoiding (by trunk and arms balance movements) logs in the river while hands-free kayaking or maneuver when cycling between people running in a park or stay standing on a surfboard when waves crash on the user. In such instances, the mechanical balance perturbations may be provided simultaneously and respectively to the cognitive balance games presented on the user screen. According to some embodiments, another game option is to connect to street view or pre-scanned environment, such that the trainee can go kayaking (low sitting position) or cycling (regular sitting position) or surfing (standing position) in a virtual environment during perturbation training, such as a hands-free bike ride in a park. In some embodiment, virtual reality (VR) glasses may be worn by the user.

According to some embodiments, feedback to the trainee’s responses and performance while perturbation training may be provided in various means, including, for example, audible feedback, visual feedback and/or tactile feedback, preferably in real time. According to some embodiments, the feedback may be voice-based feedback. Such feedback may include voice communication between the system and the trainee such that the system is configured to recognize the trainee’s voice and provide vocal feedback. For example: the user was able to answer a cognitive task presented aloud, the system recognizes his voice and marks the answer given, and also gives voice feedback on whether the answer is correct. According to some embodiments, the feedback may be tactile. In such a setting, the trainee may hold a wireless button and can respond with the push of a button in response to a cognitive task displayed on the screen/display. Cognitive reaction time and the number of correct answers may be measured, and, based thereon, a cognitive score may be provided at the end of the training session. In some embodiments, the feedback may be provided in real time, by visual feedback presented on the screen, for example, in the form of a mark or a score. In some embodiments, the feedback may be provided utilizing eye-tracker glasses configured to identity the screen location on which the subject is looking in response to a cognitive task displayed on the screen/display. Based on the location, the cognitive reaction time and the number of correct answers may be determined.

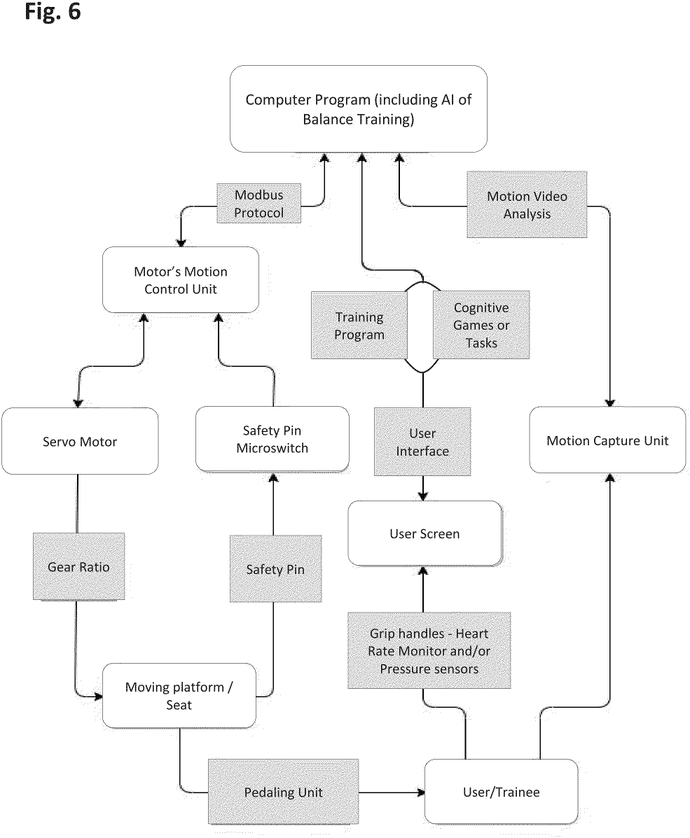

Reference is now made to FIG. 6, which illustrates communication flow chart of various component of a training system, according to some embodiments. The arrows shown in FIG. 6 represent the propagation and directions of information and data between each part and the interconnection therebetween. The light shaded boxes represent the main system parts that receive or transmit communications. The darker shaded boxes represent Intermediate elements (components) that assist with connection or communication. The flow chart shown in FIG. 6 describes the communication of the units and elements of the training system therebetween. According to some embodiments, the PerStBiRo system may include a programming mode (editing mode) and a working mode. The modes are determined by the motor’s motion control system that checks whether the safety pin micro-switch is pressed or not and sends this data to the main computer program. When the safety pin is closed (pressed micro-switch), the servo-motor does not receive electric current, and the user can edit or create a new training program or to choose the automatic computer program (AI-based program) by the user interface (on the host PC), but cannot start running it. When the safety pin is open (unpressed micro-switch), the servo-motor receives electric current, and the user may start the training program, but cannot change data on it. Thus, for running a training program, the user first creates a customized training program with the program user interface and saves it on the computer (programming mode) or chooses the automatic AI-based program for the current training session. Second, in working mode, the computer program runs/executes the training program by utilizing the motor’s motion control system and the Motion capture cameras unit. The computer program communicates with the motor’s motion control system and with the Motion capture cameras that monitor the trainee’s movements all along a training session. The motion control unit analyzes the data received from the computer program and the servo-motor to determine the next movement the motor will make, according to the training program (whether it was manually programmed or automatically programmed based on the AI computer program). For executing a perturbation command, according to the training program, the motor’s motion control system conducts electric current to the serv-motor, which is connected to the moving platform/moving seat by a suitable gear ration (such as, for example, 1:2 to 1:7, for example, about 1:6 gear ratio) to increase the applied moment on the platform/seat. The moving platform/seat, with the pedaling unit and the pedaling subject, perturbed according to the executed programmed perturbation. During the executed perturbation, once the Motion capture cameras detects an appropriate effective upper-body balance reaction (based on the AI balance training executable instructions), the platform tilt (i.e., the perturbation) is immediately stopped, and the motor returns the moving platform/seat, the pedaling unit, and the user/trainee to a vertical zero position by motor counter-rotation. In some embodiments, the trainee’s heart rate and gripping the grip handles may be monitored by sensors located on the grip handles. Some or all of the related data may be presented on the user screen throughout the program training session. After the training program, the user may review any of the related data, including, for example, video footage, moving graphs, and any of the additional data collected during a selected training session. In some embodiments, some or all of the data may be used by the computer program to determine next training sessions. In some embodiments, after the training session, the trainer may review the video footage, the trainee’s moving graphs and/or the data collected in order to determine the next training session.

According to some embodiments, the motion control system is based on the motion controller and is communicated via an interface, such as, Modbus through the computer program. The computer program utilizes the motion control unit to communicate with the servo motor, which in turn executes the perturbations of the moving platform with the STB. From the computer program, the motion controller may receive the required information of the direction, the tilt angle, maximum velocity and maximum acceleration/deceleration. The motion controller includes an internal motion profile generator that generates a trapezoidal velocity profile. Since acceleration is of great importance in providing unexpected perturbations as well as real-time implicit feedback for balance reactions, a triangular velocity profile for perturbation rotation and feedback counter rotation are utilized. The moving platform/STB accelerates for generating the required external perturbations and then decelerates to zero velocity, when a balance response is detected or not.

According to some embodiments, a motion capture unit/system (such as, a Microsoft Kinect sensors, Intel RealSense cameras or similar video motion capturing device) is associated with or integrated with the system. The motion capture unit is configured to capture in real time the body posture i.e., stick figure, and is included with the system for two main reasons. First, the motion capture unit allows to identify whether the trainee (subject) attempts to perform a reactive/compensatory balance reaction trying to straighten the PerStBiRo system to its 0° neutral position following an external perturbation. Second, the motion capture unit allows to capture throughout the trial the upper skeleton posture i.e., stick figure, including the shoulders, arms, head, and hands. Then, collect information about the trainee’s balance movements with respect to the current system state, and to analyze the trainee’s responses to ongoing events. In some embodiments, it was found that the correlations between Microsoft Kinect™ and 3D motion analysis is high for the reactive/compensatory leg movements (r = 0.75-0.78, p = 0.04). Thus, using a motion capture unit, such as the Microsoft Kinect™ system provides comparable data to a video-based 3D motion analysis system when assessing reactive balance responses in the clinic.

In some embodiments, only some of the body joints that the motion capture unit interface provides, may be used, because the lower body joints may sometime be hidden by the STB. In some embodiments, the motion information concerning such joints was occasional, less accurate, and very noisy. Given the upper body joints that were captured, several angles (α1-α6) were calculated, that were considered in the computer program: α1) Shoulders angle: The angle between the line between the two shoulders of the participant and the ground.; α2) Head-Neck angle: The angle between the line from the head to the neck of the participant and the line vertical to the ground; α3) Head-ShoulderBase angle: The angle between the line from the head to the SpineShoulder joint of the participant and the line vertical to the ground; α4) Head-CalculatedShoulderCenter angle: (Definition: CalculatedShoulderCenter lies in the middle of the two shoulders joints given by the motion capture unit.) The angle between the line from the head to the CalculatedShoulderCenter of the participant and the line vertical to the ground; α5) Left elbow angle: The angle between the line from the ElbowLeft to the ShoulderLeft joints of the participant and the line vertical to the ground; α6) Right elbow angle: The angle between the line from the ElbowRight to the ShoulderRight joints of the participant and the line vertical to the ground. One or more, or any combination of said angles may be used in the calculations.

In some embodiments, angles α1 and α2 may be used in the real-time training process for identifying the trainee’s body position with respect to the STB, in order to compute the moment an effective balance response was shown and to tilting back the STB. In some embodiments, Angels α3-α6 are not necessarily used during real-time training process but are shown in post-training graphs for advanced post-training analysis. In some embodiments, all body part locations are logged in each frame that was taken by the Kinect system for post-training calculations of these angles.

According to some embodiments, the training system includes a user interface. In some embodiments, the computer program that serves as the system’s user interface can run/be executed on a host PC. In some exemplary embodiments, the user interface application may be of a Windows format and may include three tabs: choosing and running a training program, creating a new training program, and a pop-up exiting window for saving the data captured and the post-training data and video clips tab. In some embodiments, the run training-program tab is the main tab which allows the user to choose the trainee’s training program, whether it was manually programmed, or an automatic option was selected, and starts running/executing the exercise sequence in which a series of perturbations is applied to the user/trainee. The tab allows creating or loading the trainee’s details (such as, name), opening training history is opened, selecting and loading training program (for example, from a menu). In some embodiments, the create new training program button allows opening the setting parameter tab, starting/pausing the training, selecting motion capture unit, selecting if to utilize motion feedback, controlling an emergency stop button (configured to lock the motor immediately and stops the moving platform), controlling a release brake button (which is configured to release the latch from the motor so the moving platform can freely move to all optional directions. Further optional tabs may include a screen showing the real-time frames as recorded by the motion capture cameras of the motion capture unit, marking the camera’s circle locations on the trainee’s body joints that the system identifies; a screen showing the perturbation details of the chosen training program, a list of previous and remining perturbations and marks the coming perturbation; a timer for the total time spent in the current training program; the moving seat angle, the real-time moving platform angle received from the motor; body angle, the real-time trainee’s shoulder angle that is received from the motion capture cameras system; body angle at last perturbation, the shoulder angle at the point at which the user has made the desired balance movements to trigger the system to automatically stop the perturbation; and connectivity checks for the motion capture cameras system, for the motor and moving platform/seat and for the overall system.

According to some embodiments, the creating training program tab enables creation of a new customized training program. In some embodiments, the programming of a new training program may be manual and may allow manual programming, including, for example, control of all parameters of the perturbations (magnitude, velocity, acceleration, frequency), control of the motor mode (stable or “floating” between the external perturbations), determination of block or random perturbation training, and the like. In the setting process, each perturbation may be programmed separately and added in chronological order to the list of perturbations composing the customized training program. For each perturbation, the user may set the maximal desired values of the motion profile parameters, as long as a balance response is not detected by the motion capture cameras system (acceleration, deceleration, velocity, and perturbation angle). When a balance reaction is detected, the motor will not complete its predetermined operation and, therefore, will not necessarily execute the motion profile according to the programmed maximal values. The delay time between each two consecutive perturbations, the tilting direction, and the number of perturbations during a single experiment or training may also be selected. In addition, the balance response threshold parameter may also be selected. This parameter determines at what degree of balance response the trainee will receive biofeedback in form of immediately returning the moving platform to its neutral position. This parameter is customized for each trainee based on the automatic calibration phase and by studying the same trainee’s balance response behavior based on past trainings. The result of this unique parameter is the customized training program. Accordingly, the balance response threshold parameter is the only set parameter the user/trainer can calibrate in real time while executing a training program for obtaining better biofeedback and a better motor learning process. In some embodiments, the programming of a new training program may be automatic programming. In some setting, the computer software, based on the AI previously obtained data (training), adjusts the next training session for the trainee according to the performance of the last training sessions, the frequency and duration of the last trainings while considering the date of the last training and the balance performance that are combined with the calibration data of the previous and current training session. The adjustment of the training may be made on the position of sitting or standing in the training, according to the degrees of difficulty (from low sitting - easy difficulty level, to standing position - difficult advanced level), duration of training, frequency of perturbations per minute, magnitudes and speed of perturbations, and the like, or any combination thereof.

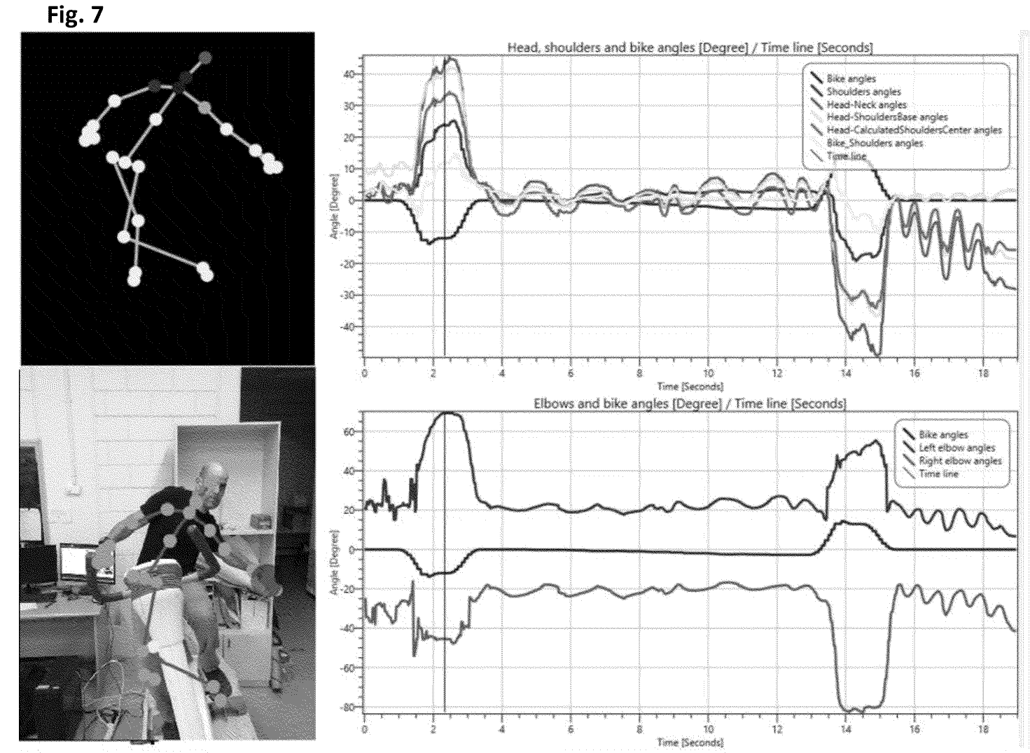

According to some embodiments, additionally user interface tabs may include, for example, saving data tab which allows the user to save or discard the training session data and video frames taken by the motion capture cameras unit. In some embodiments, a performance history tab may display training data, including, for example, how many perturbations the trainee has passed the reactive balance response threshold, graphs of the entire training and upper body responses (i.e., shoulder line movement, trunk movement and arms movements). This tab enables the trainer/user to analyze the kinematic data of a specific balance reaction in a specific training session. In some embodiments, two different kinematic graphs of the calculated angles (α1-α6) related to the moving platform/seat position in each frame taken by the motion capture cameras unit may be presented. Optionally, also presented are other diagrams, including, for example each frame of the trainee’s skeleton image (body stick figure) and of the moving seat’s position that were acquired by the motion capture cameras unit during the training session. In addition, a moveable timeline may be presented, thereby allowing the trainer to observe the α1-α6 (shoulder-line neck-line, trunk line, arms movements) angles and the body stick figure at every timestamp, compared to the moving seat’s angle position at that timestamp. In some embodiments, the computer program further allows watching the training session as a movie, optionally while pausing at specific frames. Exemplary presentation of a history tab of a user interface, displaying training data of a training session is shown in FIG. 7.

According to some embodiments, the control unit of the system includes one or more processors (for example, in the form of host PC) that are configured to execute a computer program (executable instructions) which is configured to control operation of the training system and, inter alia, provide real time feedback to the subject regarding reactive balance reactions and/or success in cognitive tasks and/or to determine the next training session. In some embodiments, at least some of the computer program include machine learning and Artificial intelligence (AI) algorithms, which can: provide the trainee real time feedback to reactive balance reactions and/or to success in the cognitive tasks and/or B) builds the next balance training.

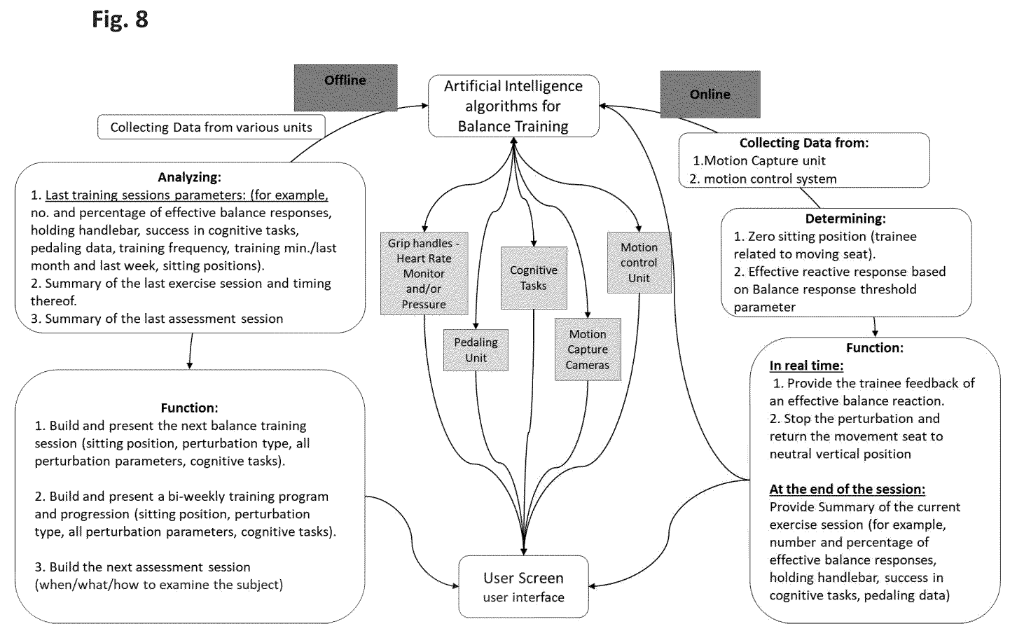

Reference is now made to FIG. 8, which is a flow chart of steps of AI algorithms utilized in balance training system, according to some embodiments. The AI algorithm utilized includes online processing and/or offline processing. According to some embodiments, the online processing may be executed by a local processing unit. In some embodiments, the online processing is configured to provide the trainee with real time feedback to reactive balance reactions during balance exercise. Firstly, calibration stage is performed (for example, in the length of 60-180 seconds, such as, for example, 90 seconds). At the end of the calibration stage, the AI computer program and the motion capture cameras unit can automatically customize the PerStBiRo system to the current trainee by calculating: 1) the individual upper body sway amplitude during pedaling without perturbations, and/or 2) the Trainee-Moving seat zero point. Next, during the customization process, the shoulder line angles relative to the horizon and neck-line angles relative to vertical line are recorded separately for a period of time (for example, 60-180 seconds, such as, 90 seconds), during the second part of the calibration stage, that can last for example, for a total of 2-5 minutes (such as, 3 minutes). At the end of this stage, the individual upper body sway amplitude and the Trainee-Moving Seat zero point are calculated for both angles (shoulder line and neck-line). Thereafter, the angles that show more stability and less noisy parameters are automatically selected to be the angle on which the AI algorithm (software) relies on, in order to set the balance threshold parameter and to provide the real-time sensorimotor feedback for an effective balance reaction, by returning the moving Seat (i.e., seat on the moving platform) to its vertical position. Thereafter, during balance exercise stage, the trainee is exposed to a variety of repeated random unexpected perturbations, as detailed above herein. When each of the perturbation is executed, the AI computer program analyzes (checks) the difference between the trainee’s upper body angles (shoulder line and neck line) and the Moving Seat angle and takes into account the body amplitude of the user and the Trainee-Moving Seat zero point to determine if a significant balance reactive recovery response rather than a regular paddling movement was detected. In case the PerStBiRo system detected an effective reactive balance reaction (higher than the balance threshold parameter), that was identified by the motion capture cameras system, the moving seat tilt rotation (i.e., the perturbation) is stopped immediately, and the motor returns the moving seat to its vertical position (i.e., its neutral/zero position) by motor counter-rotation. At the end of the current training session, a summary of the current exercise session may be provided to user. The summary may include various parameters, including, for example, number and percentage of effective balance responses, number and percentage of time the subject held the handlebar for assistance, success in cognitive tasks and/or pedaling data (such as, time, distance and set resistance). At the end of the current training session, the next training session may be presented. In some embodiments, a training program (for example, bi-weekly program) may also be presented.

According to some embodiments, the offline processing may be performed on a database server (local or remote server). In some embodiments, after each training session the AI program is configured to collect all or at least part of the data from the motor’s motion control system, motion capture unit, pedaling unit, grip handles (heart rate monitor and/or pressure sensors), Cognitive tasks, and the like or any combination thereof. Based on the collected data the AI program can analyze one or more of:

1. Last training sessions parameters: number and percentage of effective balance responses, wherein only if the percentage of effective responses is over a predetermined threshold (for example, over 80% effective responses), the AI algorithm may offer the next level of perturbation training (otherwise the same level of training is offered); number and percentage of time that the trainee held the handlebar, wherein only if the percentage of time of hands free pedaling is determined (for example, over 80% of the time with hands-free pedaling) the AI program may offer the next level of perturbation training (otherwise the same level of training is offered); number and percentage of successful cognitive tasks answer and games wherein only if the percentage of correct answers and/or correct movements during execution of the cognitive tasks are correct (for example over 80% correct answers and correct movements during cognitive balance tasks), the AI algorithm may offer the next level of cognitive tasks (otherwise the same level of training is offered); pedaling data (including, time, distance, resistance); training frequency; training min./last month and/or last week; sitting positions in the last training session; and the like, or any combination thereof; 2. Summary of the last exercise session and when it was executed; 3. Summary of the last assessment session. Based on at least part of such data, the AI algorithm is configured to build and present the next balance training session (sitting position, perturbation type, all perturbation parameters -magnitudes, velocities, accelerations, frequencies and type of perturbations, level of cognitive tasks); 2) Build and present a bi-weekly training program and progression (sitting position, perturbation type, all perturbation parameters- magnitudes, velocities, accelerations, frequencies and type of perturbations, next cognitive tasks, pedaling resistance) and/or 3) Build and notify the next assessment session (when/what/how to examine the subject (by himself and/or by a trainer).

According to some embodiments, the data collected and/or analyzed by the system may be used for training the machine learning models. In some embodiments, data collected from various training systems may be used in the training processing. In some embodiments, the data collected from a plurality of training systems (each may be located in a remote location) may be stored and/or at least processed on a remote server (such as, a cloud-based server, or any type of server). In some embodiments the training system further includes a communication unit. In some embodiments, the remote server may be functionally associated with the control unit of the system via the communication unit.

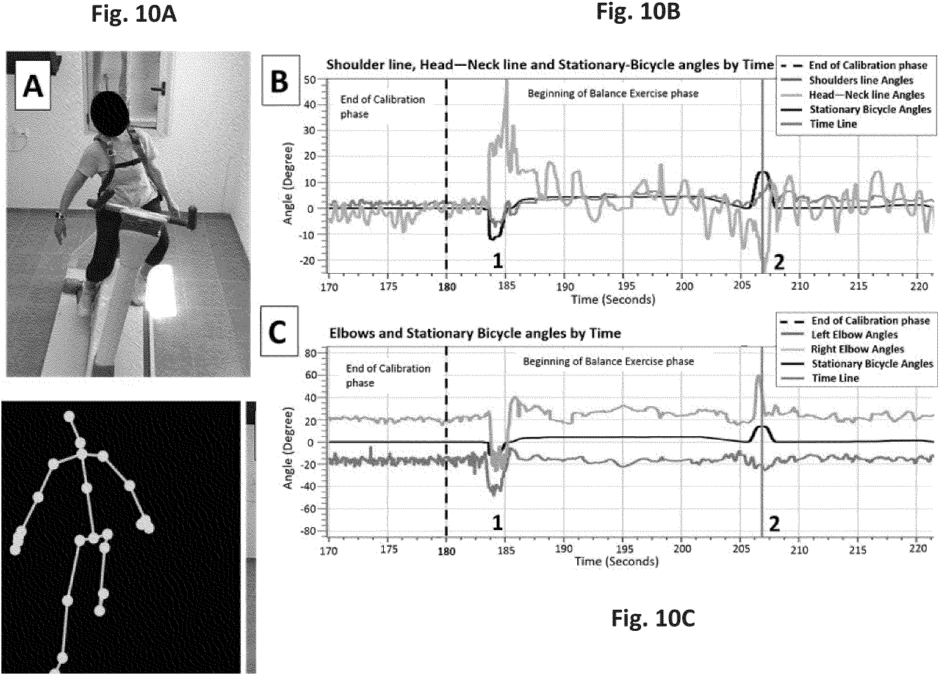

Reference is now made to FIGS. 10A-C, which shows the implementation of the motion capture unit-system function during training process. FIGS. 10A-C presents a short sample of the motion capture unit during a training process. In each training session, (in older adults usually lasts 20 minutes), there are two stages: 1) calibration stage (the first 3 minutes) and 2) balance exercise stage (17 minutes). 1) The calibration stage is for minimizing errors in the motion capture unit calculations and for automatic customizing the PerStBiRo system to the participant who is using it currently. It consists of two parts: A) trainee’s adaptation phase - 90 seconds of slow pedaling in order to let the subject ease into a comfortable position. In this phase the computer program does not make any reference point calculations due to the noisy data that was gathered by the motion capture unit before the subject has fixed his sitting. B) Measuring and calculating the individual upper body sway amplitude (the body-sway base noise) and the trainee-STB zero point -90 seconds of self-paced pedaling while the Microsoft Kinect™ system (or other motion capture units) provides data to the computer program for calculating angles α1 and α2 as described above. At the end of the calibration stage, the computer program calculates customized reference angles that are used later to determine whether the trainee’s balance reaction is effective or not. Based on the identified minimal body sway amplitude, the computer program detects if the participant responded to a given perturbation or whether their current body angle is a part of natural movement during paddling (i.e., into the body-sway-base-noise range). Thus, firstly the computer program records the data of the α1 and α2 angles during the 90-seconds calibrated self-paced pedaling. Secondly, it calculates separately for each angle (α1 and α2) the upper body sway amplitude and the trainee-STB zero point. The amplitude of each angle (α1 and α2) may be approximated by using the formula: {Max (angles) - Min (angles) }/2, where angles are the list of all the angles that were recorded. The trainee-STB zero point is the angle the participant is naturally sitting on the STB during paddling and is necessary because often older adult trainees naturally tend to lean a few degrees to either the left or the right side. Thirdly, based on the least “noisy” angle during calibration stage α1 or α2), the computer program select automatically to use its upper body sway amplitude and the trainee-STB zero point to determine whether the movement that the participant has made is a reaction to the perturbation or it is just the effect of paddling. 2) The balance exercise stage (about \17 minutes for older participants) contains randomly and unexpected balance perturbations. When a new perturbation is executed, the computer program checks the difference between the participant’s angle (calculated by the chosen formula, α1 or α2 following the calibration stage, see FIGS. 10B, 10DD) and the STB’s angle (computed using the trainee-STB zero point of α1 or α2, and the participant as an anchor for this calculation) and takes into account the body amplitude of the participant to see if there has been a significance movement rather than a paddling movement.

According to some embodiments, two options for separating a recovery balance reaction to a normal paddling movement following a perturbation were programmed: Option A - checking if the STB is leaning in the opposite direction to the body movement, above a threshold angle. The threshold is the summation of the trainee’s body amplitude and a programmed predetermined bias. This additional selected bias requires the trainee for a larger distinct balance reaction to recover their balance for passing above the response threshold and stopping the perturbation by turning the STB back to its zero position. This option deal with subjects who exhibit large body amplitudes during the exercise session versus calibration phase. This summation threshold is denoted “diffAllowed”. If the STB and the trainee’s body are leaning in opposite directions, the computer program checks if the current body angle is larger than [trainee-STB zero point + (diffAllowed / 2)]. If so, then the participant performed both, moved in the opposite direction of the STB and passed its trainee-STB zero point. Then the command to stop the perturbation and return the STB will come out. Option B - checking if the participant is leaning to the opposite direction of the STB, regardless of the trainee-STB zero point.

According to some embodiments, the training process includes recording the data obtained by the motion capture unit skeleton joint identification and capturing RGB frames for post-training analysis. The post-training analysis application displays a movie of the trainee riding the PerStBiRo system and kinematic graphs that show the α1 and α2 angles (α3-α6 angles can be displayed by choice) at every timestamp, compared to the angle of the STB at that timestamp. This allows the physical therapist/trainer to determine how to proceed with the training in the next sessions, and also allowing the researchers to analyze the trainee’s balance behavior to the perturbations. In some embodiments, as detailed herein, the post training analysis is performed by computer executable instructions.

According to some embodiments, there provided a post-training analysis software: which enables the trainer or subject to analyze the kinematic data of a specific trainee in a specific training session. For example, two different kinematic graphs of the calculated angles (α1-α6) related to the STB position in each frame taken by the motion capture unit may be displayed. This window can also present two other diagrams: the trainee’s skeleton image (body stick figure) and the STB’s position that were taken by the motion capture unit, each frame during the training session. In addition, a moveable timeline is presented and allows the trainer to observe the α1- α6 angles and the body stick figure at every timestamp, compared to the STB’s angle position at that timestamp. The software also allows the trainer to watch the training session as a movie, and to pause at a specific frame.

According to some embodiments, safety is an important issue since unexpected perturbation are applied to older adults and may cause then falling of the PerStBiRo system. During the PerStBiRo exercise session the moving platform is perturbated unexpectedly to challenge their balance. The subjects are instructed to recover from the perturbations using upper body movements as fast as they possibly can, which is the most important part of the training regimen. The motion tracking unit tracks their recovery balance movement responses (for example, side bending of the trunk) to result in platform movement back to the starting position. In a study conducted by the inventors, it was found that young individuals respond by trunk response to the opposite direction of the perturbation to quickly move the upper bodies’ center of mass toward the base of support provided by the STB siting chair. In case the subject fails to recover and falls from his/her chair, the patient is wearing a safety harness that can arrest the fall before the patient’s fell of his/her chair. Examples of such a safety harness are the Skylotec G-0904 or the PN12 harness. The safety harness may be hung from the ceiling by two ropes above the patients (for example, FIG. 1), or may be hung from an arm of the system (for example, shown in FIGS. 4A-D). For stability reasons the ropes do not necessarily hang straight from the ceiling or arm, but in a diagonal such that the distance between the connection points of the two ropes on the ceiling or arm is about 2 m. When the rope is hanged in diagonal it is capable to apply much larger horizontal force in order to keep and stabilize the patient at the center.

According to some embodiments, the training system disclosed herein may be portable. In some embodiments, the training systems may be for home use, by a subject. In some embodiments the training systems may be for institutional use.

In some embodiments, the system can be self-operated by a subject, for example, in home use. In some embodiments, for such home operation, the trainer station can be moved, so that the user interface is displayed directly on the system’s display. Thus, the trainee by himself/herself operates the system and can trains on the system on his own.

According to some embodiments, the system and methods disclosed herein allow implicit learning of the subject to improve balance.

According to some embodiments, the systems and methods disclosed herein advantageously allow dual tasking training, whereby both a physical training (perturbations) and mental training (cognitive challenge/stimulation (for example, by cognitive task and/or cognitive games) are provided to the user and the performance of the user is further configured to be assessed by the system. The dual tasking training, may synergistically act in consequently improving the subject’s balance.

In some embodiments, the machine learning algorithms disclosed herein can advantageously be utilized to assess the performance (physical and mental), provide real time feedback to the user, adjust the training level and/or determine a training session.

According to some embodiments, there is provided a mechatronic bicycle simulator system which includes: a stationary training bicycle (STB); a moving platform; one or more motors; and a central control unit; wherein the moving platform is configured to provide perturbation tilts to the STB, thereby stimulating balance of a user situated on the STB.

According to some embodiments, the system may further include a gear mechanism.

According to some embodiments, the gear mechanism may be configured to allow the transmission of the motor rotation with the moving platform rotation axis by one or more ball bearings, thereby allowing the moving platform rotation and the balance perturbation tilts.

According to some embodiments, the tilt angle is up to 20 degrees to each side. According to some embodiments, the system is configured to allow an acceleration and deceleration of about 30 m/s^{^2}. According to some embodiments, the system is configured to allow a velocity of about 30 m/s.

According to some embodiments, the system may further include a motion control unit.

According to some embodiments, the system may further include a motion capture unit. In some embodiments, the motion capture system comprises a Microsoft Kinect Unit™.

According to some embodiments, the central control unit is configured to control the motion control unit and/or the motion capture unit.

According to some embodiments, the central control unit comprise a processing unit configured to execute a computer program (executable instructions).

According to some embodiments, the system may further include a user interface and/or a display.

According to some embodiments, the motion control unit may be configured to direct the motor rotation, based on a predetermined (programmed) training plan.

According to some embodiments, the training plan may include a triangular motion profile for each perturbation (acceleration-deceleration).

According to some embodiments, the central control unit may allow a trainer or the subject to determine the training plan and control one or more balance exercise parameters.

According to some embodiments, the balance exercise parameters may be selected from: maximum acceleration/deceleration, maximum velocity, angle of perturbation, number of right/left perturbation repetitions, the delay time between the perturbations, or any combination thereof.

According to some embodiments, the processing unit may be configured to provide a real-time feedback to the user of a trainee’s reactive balance reaction following a perturbation.

According to some embodiments, the perturbations provided are expected or unexpected.

According to some embodiments, there is provide a method for training balance of a subject, the method includes the steps of:
situating the subject on a STB of the system disclosed herein;
harnessing the subject;
providing one or more perturbations to the STB; and
detecting the subject reactive balance reaction following a perturbation.

According to some embodiments, the method may further include providing a feed-back to the user.

According to some embodiments, the method may further be configured to adjust operating parameters of the training session based at least in part on the analyzed balance response.

According to some embodiments, method may be further configured to determine or recommend operating parameters of a following training session and/or a training plan, comprising two or more consecutive training sessions.

According to some embodiments, there is provided a non-transitory computer readable medium storing computer program instruction for executing the training method disclosed herein.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system’s registers and/or memories, into other data similarly represented as physical quantities within the computing system’s memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1: Exemplary Components of a PerStBiRo System

An exemplary PerStBiRo system (as illustrated in FIG. 1) weights about 90 kg and the hardware includes the following components: moving-platform frame, stationary frame, STB, servo motor, motion control system, gears and gear mechanism, two ball bearings and Kinect motion capture unit. The details of the components are listed in Table 1.

TABLE 1

| | | |
|---|---|---|
| Component | Manufacturer | Model |
| Ball bearings | NSK | Pillow Block UCP210D1 |
| Motion Controller | Emerson | Epsilon EP202-P00-EN00 |
| Stationary training bicycle | BioCor | UM-3296 |
| Servo Motor | Emerson Control Techniques | Unimotorfm model 095E2C300VBCAA100190 |
| Kinect camera | Microsoft | |

### Example 2- Testing the Effects of Bicycle Simulator Training System on Anticipatory and Compensatory Postural Control in Older Adults

### Methods and Analysis

The example describes a parallel single-blinded randomized-controlled trial (RCT) that follows the recommendations of SPIRIT 2013. Older adults are randomly assigned to one of two groups: 1) Perturbation Training during hands-free Stationary Bicycling Riding (PerTSBR), or 2) Training of Stationary Bicycle Riding without perturbations (TSBR). Both groups are trained in a sitting position by the same PerStBiRo system (illustrated in FIG. 1), twice a week for 12 weeks, (details in regard to the magnitude and progression of the balance training is presented in Table 2. Compensatory (reactive) and anticipatory (proactive) balance control during standing and walking, functional balance, function, fear of falling, and aerobic endurance are measured pre- and post-training at the Schwartz Movement Analysis & Rehabilitation Laboratory in the Physical Therapy Department at the Faculty of Health Sciences at Ben-Gurion University of the Negev, Israel. The intervention is being provided inside participants’ community centers or in their protected housing.

Participants: A convenience sample of 68 community-dwelling older adults are recruited. Eligibility criteria are: 70 years of age or older, ability to walk independently without assistive devices, independence in daily living activities, and provision of a medical waiver signed by their primary care physician allowing participation in physical training that requires pedaling on a stationary bicycle two or three times a week. After completing a medical history questionnaire via an in-person interview at enrollment, volunteers are excluded if they suffer from: (a) ischemic heart disease which restricts exercise, (b) chronic obstructive pulmonary disease, (c) uncontrolled blood pressure, (d) severe vision problems (blindness), or (e) cognitive impairment, scoring less than 24 on the Mini-Mental State Examination. Further exclusion criteria include: (f) a period less than one year after hip or knee replacement surgery or after fractures of the lower extremities, (g) amputation of a lower limb, (h) neurological diseases or after a stroke, and (i) inability to ambulate independently, and/or (j) weight > 120 kg (exceeds safety harness weight limits).

The Interventions: Trained physiotherapists conduct individualized training. They received training to operate the PerStBiRo system (shown in FIG. 1), and experienced it themselves to be able to determine the challenge for a participant and adjust the difficulty of the training. The same PerStBiRo system is used to train both the PerTSBR and TSBR groups. Each group receiving 24 training sessions, twice a week for 12 weeks.

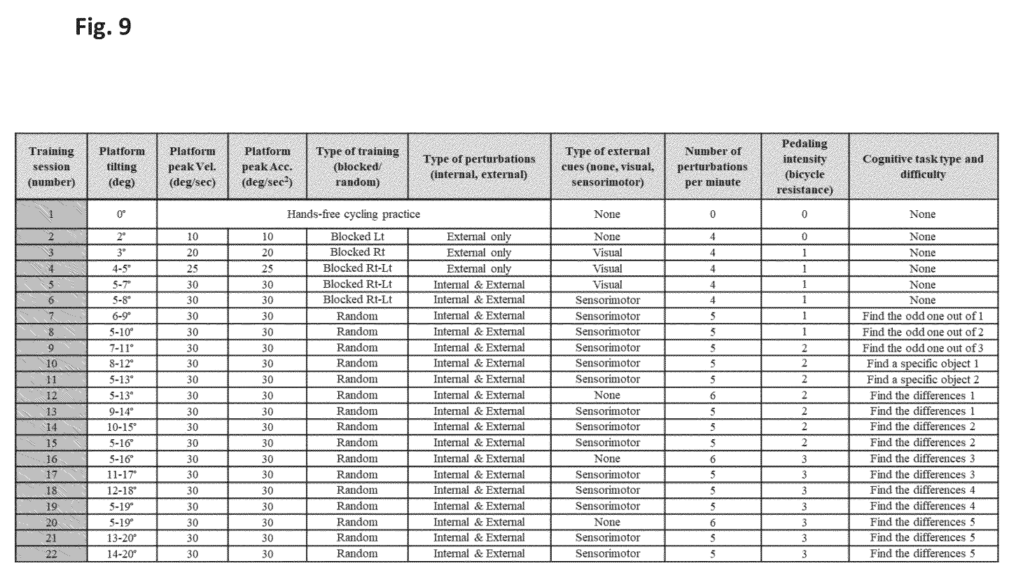

Each session lasts for 20 minutes and includes three parts: stage 1) warm-up—3 minutes of self-paced pedaling with the same bicycle resistance for both groups, corresponding to the training program level, without perturbations and cognitive tasks; stage 2) main exercises—15 minutes of perturbation training during hands-free stationary bicycling (PerTSBR). For details, see “Experimental group intervention”, with perturbations and “Control group intervention” without perturbations (TSBR) in combination with concurrent visual cognitive tasks for both groups. The graduated difficulty levels in bicycle resistance and the cognitive tasks are the same for both groups and determined according to the training program (details in the table presented in FIG. 9); and stage 3) cool down—2 minutes of self-paced pedaling without bicycle resistance, without perturbations and cognitive tasks. During stage 2 the participants are instructed to “ride the bicycle at your preferred pace and try to stabilize yourself. Try also to do your best in the cognitive tasks”. The cognitive dual-task exercises are presented using Microsoft Power Point on a screen 2.5 meters in front of the pedaling trainee’s head. It includes tasks such as “Find the odd one out”, “Find a specific object in the picture”, and “Find the differences” between two pictures. For all tasks presented, the participant must state out loud where the object is, then have this checked by the trainer, and if the answer is correct, the next task is presented. During the training sessions trainees wear a loose safety harness that can arrest falling using the PerStBiRo system, but still allow comfortable pedaling and execution of balance recovery reactions with the upper body without the harness suspension. In both groups, each participants activities are documented in each session.

Experimental group intervention: The PerTSBR experimental group participants receive a combination of internal and external medio-lateral (ML) tilting perturbations during hands-free bicycling under dual-task conditions. This is provided by roll-angle (tilt) balance perturbations that aim to evoke trunk and arm balance recovery responses. The tilt pivot axis is lower than the trainee’s Center of mass (CoM) that is located in the pelvis above the bicycle seat. Therefore, when tilting the trainee’s CoM aside rapidly, the trainee is forced to respond reactively with trunk and arm movements. The PerStBiRo system provides a maximum right and left perturbation tilt angle of 20° (each side) with maximum acceleration and deceleration of 30 m/s2 and maximum velocity of 30 m/s, which is usually that of a triangular motion profile for each perturbation (acceleration--deceleration). The computer program allows the therapist trainer to determine the perturbation training plan and control all balance exercise parameters: maximum acceleration/ deceleration, maximum velocity, angle of perturbation, the number of right/left perturbation repetitions, and the delay time between each pair of consecutive perturbations. Diagnosis of an effective balance response to each trainee following unannounced balance perturbation is the basis for providing the trainee with real-time customized sensorimotor cues. These cues lead to improvement in the trainee’s internal sensorimotor feedback of successful trunk and arm balance recovery reactions and, therefore, to better implementation of motor learning of balance reactive control. Thus, a calibration phase is needed. During the calibration phase (FIGS. 10B and 10C, left of the black dotted lines), by capturing the upper body joints, the computer program calculates two angles: 1) the shoulder line angle—the angle of the participant’s shoulder line and the ground (FIG. 10B, horizontal purple line), and 2) the head-neck angle—the angle of the participant’s head to neck line and the vertical line to the ground (FIG. 10B, horizontal green line). The sequence of angles that maintains more stability and less noisy singles are automatically selected as the key-factor angles that the program relies on in determining the trainee’s sitting posture and differentiating upper-body oscillations during pedaling (FIG. 10B, calibration phase, horizontal purple and green lines) from significant reactive balance responses following external perturbations later into the training session (FIG. 10B, balance exercise phase, horizontal purple and green lines at points where the horizontal black line shows humps). Then, relying on this key-factor angle, the real-time sensorimotor cue to an effective balance reaction is given by returning the PerStBiRo system’s training bicycle to its vertical position. For example, in FIG. 4, the trainee was exposed to a programmed 20° right tilting perturbation at the 206th second of the training session, indicated by the gray timeline in 4B and C. However, an effective sharp and large upper body balance response (FIG. 10A) had already been identified when the PerStBiRo system’s training bicycle was at about 14° (FIG. 10B, the hump on the horizontal black line); thus, the perturbation was immediately stopped, and the device was returned to its vertical position. In addition, the elbow angles are also recorded for monitoring arm reactions (FIG. 10C).

All perturbation parameters, as well as the trainee’s shoulder line angles, head-neck angle, and elbow angles are displayed on the host-PC screen in real time. Also, the angle of perturbation as soon as an effective balance response is detected by the motion capture unit (Microsoft Kinect™ system) is displayed in real time. Thus, the therapists can compare the programmed perturbation angle with the actual perturbation angle (once an effective balance response is detected) and monitor the patient’s ability to recover from perturbations along the training session.

During the first stage of the training session, i.e., the warm-up phase, the PerStBiRo system calibrates the trainee’s upper body movements and their body configuration and position. The customized calibration is performed in the same fixed mode or unfixed and unstable “floating” mode as the PerStBiRo system is expected to be in during a specific training. Fixed mode is when the PerStBiRo system is locked vertically and used as a regular stationary bicycle, while the “floating” mode is when the moving platform is unfixed and unstable, floating like a surfboard and is subjected to the forces acting upon it by the pedaling trainee. Calibration is necessary to identify an effective reactive balance response later in the training session. In the second stage of the training session, i.e., the main training phase, has internal and unannounced external balance perturbation exercises in self-paced hands-free pedaling under dual-task conditions. Pedaling the PerStBiRo system in its unfixed and unstable “floating” mode causes self-induced perturbations similar to outdoor bicycling, and challenges proactive balance control. These internal perturbations are initiated from training level 5. The programmed unannounced external perturbations are ranged from low to high controlled, unexpected machine-induced ML tilting perturbations, which evoke fast upper body reactive balance responses (i.e., trunk, hip, head, and arm movements). These perturbations can be programmed and delivered as a block or random (in onset, magnitude, and direction) type of training. During perturbation exercise, the PerStBiRo system provides trainees with real-time visual or sensorimotor external cues (cues are given from training level 3).: 1) A visual cue for a beginner trainee is obtained by self-watching their balance performance in real-time on a screen, (like a mirror view). 2) For an advanced trainee, a sensorimotor cue is obtained by providing a real-time sensorimotor cue to the trainee’s balance reaction following a perturbation. Once an unexpected balance perturbation is given, when an appropriate balance reaction is detected, the tilting platform (the perturbation) is stopped immediately, and the PerStBiRo system returns to its vertical position (. This sensorimotor cue leads to improve the trainee’s internal sensorimotor feedback, therefore, to better implement motor learning of reactive balance control. In addition, concurrent visual cognitive tasks are displayed on the PerStBiRo system’s screen. The third stage of the training session, i.e., the cool-down part, includes self-paced pedaling without bicycle resistance, and without cognitive tasks and external programmed perturbations. However, the fixed or “floating” PerStBiRo system mode remains as it was during a specific training, so the trainee may be exposed to self-induced internal perturbations.

The difficulty of the perturbation training level is adjusted according to the trainee abilities, starting from the lowest level at the first training session. If the trainee is able to recover from all perturbations during the session (i.e., one did not hold the bicycle handlebars or fall into harness system during the session and feels that they can be further challenged), a higher level of perturbations will be introduced in the next session. in case the trainee was not able to recover, the same level of perturbations is introduced again until the participant can successfully recover balance in the entire session. Assistance and support are provided for trainee who feel uncomfortable in the initial phase of the training. However, they are encouraged to perform exercises with no or minimal external support.

The training program (FIG. 9) includes 22 potential training levels, which contain a gradual increase in difficulty in several exercise components with respect to motor learning, strength, endurance, and especially balance control: 1) the training starts with hands-free cycling practice to avoid external support on the handlebars that significantly reduces the postural responses and to calibrate the software to identify balance recovery responses; 2) the perturbation magnitude increases (i.e., increases displacement, velocity, and accelerations of the tilting translations); 3) the type of training shifts from the block PBBT method at the beginning, where the participant is aware of the direction of perturbation (right-left-right etc”), with a similar time interval between perturbation, thus these are expected perturbations. Than announced random PBBT was introduced (random in onset, direction, and tilting magnitude of the perturbation); 4) the type of perturbation begins with external perturbations only when the simulator system is fixed and stable in the first sessions and changes to a combination of external and internal perturbations that are the unfixed and unstable surface which is affected by the forces exerted on the platform while the trainee pedals in increasing intensity; 5) the external cue (feedback) type also changes from no cue at all to a visual cue that is like real-time viewing of a mirror on a screen during exercise and then changing to an external sensorimotor cue that leads to improving the intrinsic sensorimotor feedback. Once an unexpected balance perturbation is given, when an appropriate balance reaction is detected, the tilting perturbation is stopped, and the simulator system returns to its vertical position. This intrinsic task feedback provides the learner (trainee) with an implicit cue for successful balance response and provides the best possible motor learning implementation. In addition, at training levels 12, 16, and 20, no external cue is given in order to maximize the subject’s upper body movements to improve their upper body range of motion (especially of the trunk); 6) the pedaling intensity; and 7) the cognitive tasks difficulties also increase along the training process.

Control group intervention: The control group (TSBR) receives 20 minutes of bicycle riding on the PerStBiRo system in its fixed mode along all of the training program (used as a regular stationary bicycle) without any internal or external perturbations, but dual-task training is provided. Pedaling intensity and cognitive tasks are the same as in the intervention group and follow the levels of difficulty that are reported in the training program (FIG. 9). This training method was chosen in order to match all other training components (i.e., session time and training period, and pedaling and cognitive demands), except for the balance challenge.

Primary outcome measures - Because this study deals with the ability to learn and generalize reactive balance responses that are acquired in a sitting position into a target context of fall risk factors and balance control performances in standing and walking situations, the primary outcome measures to evaluate the effect of PBBT during bicycle riding are derived from the ability to recover from unexpected external perturbations in the compensatory step execution tests during standing and walking. The best observational and kinematic measures to represent the reactive (compensatory) balance responses are: the single-step and multiple-step thresholds (observational parameters), and the first-step recovery initiation duration, first step duration, total balance recovery duration, and total CoM displacement (kinematic parameters). Observational analysis: single-step and multiple-step threshold levels for AP and ML directions following a loss of balance are verified using a Vicon Motion System (Oxford, UK), allowing image pauses, slow motion, and running of the image back and forth. The single-step threshold level is defined as the minimum perturbation magnitude that consistently elicited a single compensatory step for at least two consecutive perturbation magnitudes. The multiple-step threshold is defined as the minimum perturbation magnitude that consistently elicited a sequence of recovery steps. During the analysis, the step recovery strategies are explored during all single-step and multiple-step trials during the whole protocol. Kinematic analysis: 3D kinematic data are collected through optical motion capture (Vicon Motion Systems, Oxford, UK), providing kinematic analysis of a motion sequence.

Secondary outcome measures - Secondary outcomes include balance and aerobic endurance measures that are assessed pre- (T1) and post-(T2) intervention by the following tests: 1) The Postural Stability Test measures balance postural control by the body sway in upright standing; 2) The Voluntary Step Execution test measures anticipatory (proactive) balance control by the ability to perform a quick step; 3) The Berg Balance Scale (BBS) is a clinical functional balance instrument that assesses balance and fall risk; 4) The Six-Minute Walk Test (6MWT) is a sub-maximal test of aerobic capacity measuring the maximum distance that a person can walk in six minutes; The Late Life Function and Disability Instrument (LLFDI) which is a self-reported function measuring difficulty in performing basic and advanced daily physical tasks; 6) The Falls Efficacy Scale-International (FES-I) which evaluates fear of falling while performing indoor and outdoor social and physical daily activities.

### Results

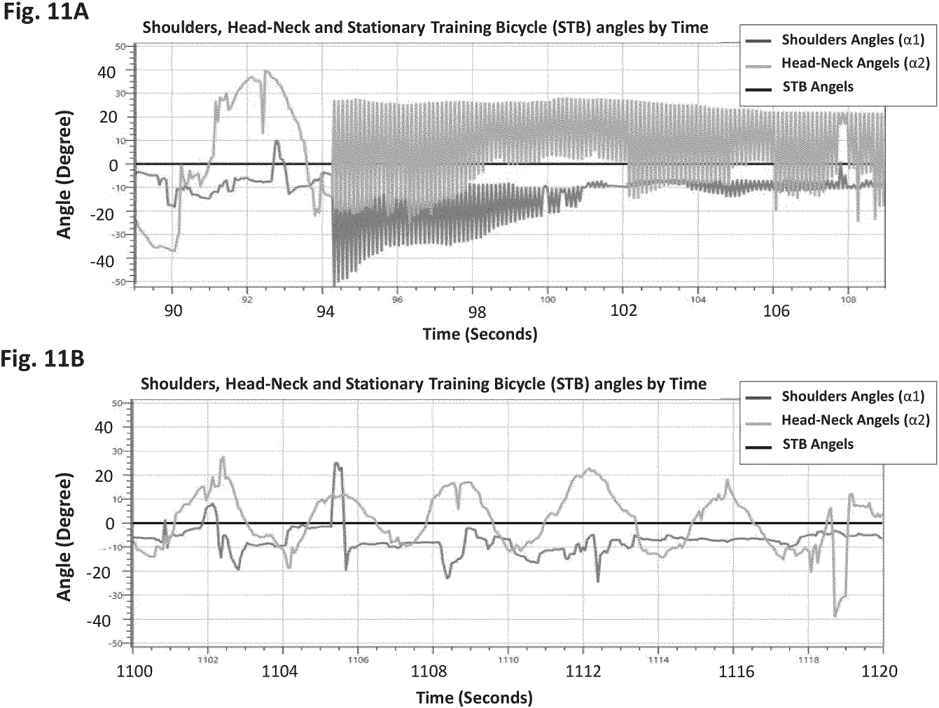

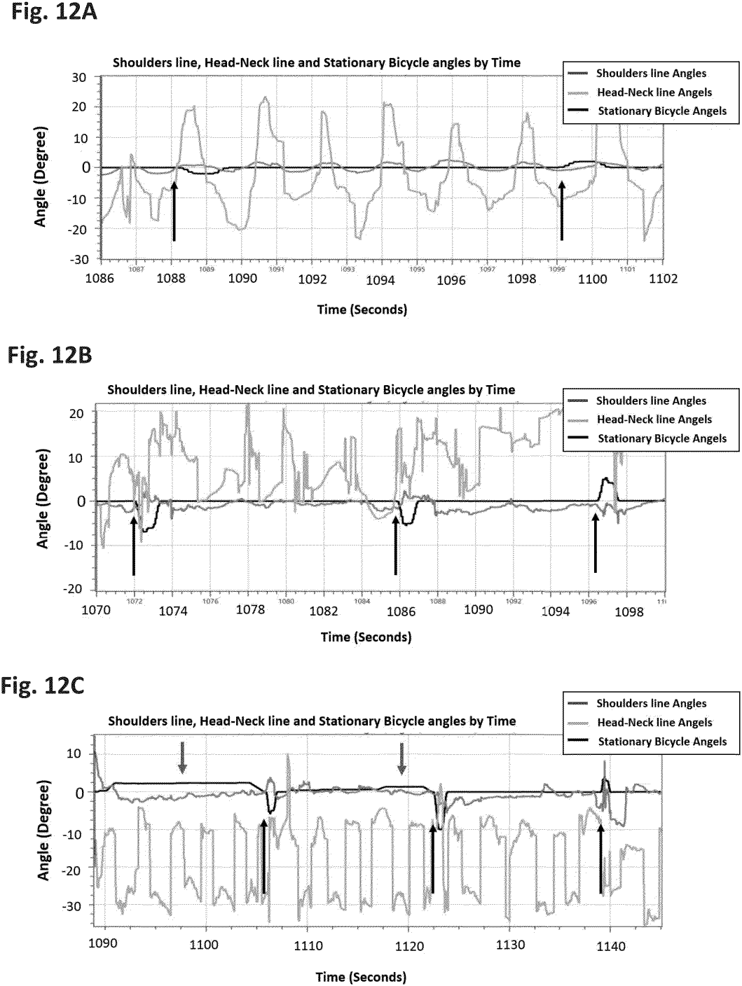

Presented herein are the results of a feasibility study to explore the ability of 86-years-old subject to train and react effectively to unannounced/unexpected perturbations during 14 training sessions of bicycling on the PerStBiRo system with increasing perturbations magnitudes. The 86-years-old subject reported few falls in the past six months, with high fear of falling. His upper-body balance reactive responses are presented by the shoulder line and head-neck angles. These parameters presence of an upper-body balance reactive response. The skill acquisition and motor learning of the 86-year-old male during the training is demonstrated in FIGS. 11A-B and FIGS. 12A-C.

In the first training session, the participant pedaled with, and then without, holding the bicycle handlebars (i.e., hands-free) for 20 min without perturbation. Exercises in this session represent little actual challenge to the balance control system. The goal of this training session was mainly directed towards a cognitive understanding of the exercises, and an improvement in self-confidence for training at higher levels. FIG. 4 shows the PerStBiRo software’s ability to monitor and identify the process of the participant’s sensorimotor adaptation to hands-fee pedaling. The first phase is to release the hands from the handlebar about 94 seconds into the training, which causes massive and noisy upper-body movements (FIG. 11A). The second skill acquisition phase is demonstrated by very organized and reduced upper-body movements (FIG. 11B) by the end of the first session. This represents an improved balance control ability.

In FIG. 12A, an example from the 3rd training session where the participant was exposed to low magnitude, velocity and acceleration in low frequency of announced perturbations (2.5° tilt) in block right-left training during hands-free pedaling. FIG. 12A shows that that despite pedaling without external support (hands free), the subject reactively responded well when exposed to unannounced balance perturbations. The tilting perturbation evoked balance reactive trunk, head, and arm movements always in the opposite direction of the perturbation to quickly move the upper body’s CoM toward the base of support provided by the stationary-bicycle seat (FIG. 12A). At the beginning of the training session, the PerStBiRo software was unable to accurately identify balance reactive responses, although the participant proactively and reactively responded to balance these perturbation responses. Later in the same training session, the PerStBiRo software able to detect proactive and reactive balance responses and, thus, control the PerStBiRo motors.

In the eighth training session (FIG. 12B), the trainee was exposed to random moderate magnitudes, velocities and accelerations of unannounced external perturbations (6°-10° tilt) during hands-free pedaling. FIG. 12B shows an increase in the older subject’s ability to response reactively to perturbations of varying difficulty, and also that the PerStBiRo software is able to monitor and recognize reactive balance responses and to provide the trainee with sensorimotor feedback of his effective responses. Although this in the eighth training session, the magnitudes of perturbations were increased the trainee’s balance reactive responses. The subject was capable of contract responding to with these challenges effectively, most likely probably due to his past experience.

In the fourteenth training session (FIG. 12C), the subject was exposed to random high magnitudes, velocities and accelerations of unannounced external perturbations (8°-12° tilt) that were provided during hands-free pedaling in the unfixed and unstable mode of the moving platform (the “floating” mode). FIG. 12C demonstrates that the proactive (indicated by red arrows) and reactive (indicated by black arrows) upper-body balance responses were appropriate most of the time for the diverse challenge of the perturbations, and therefore, reflect an effective motor learning of upper-body balance reactive responses that was acquired over the past training sessions.

Conclusion: the results presented herein show that balance reactive responses can be evoked and improved by older adults and that the older subject was able to train in an advanced training session (8th and 14th sessions) and still react effectively to higher levels of perturbations.

### REFERENCES

Anderson-Hanley C, Arciero PJ, Westen SC, Nimon J, Zimmerman E. Neuropsychological benefits of stationary bike exercise and a cybercycle exergame for older adults with diabetes: an exploratory analysis. J Diabetes Sci Technol . Jul. 1, 2012 ;6(4):849-57.

Bergen G, Stevens MR, Burns ER. Falls and Fall Injuries Among Adults Aged ≥65 Years - United States, 2014. MMWR Morb Mortal Wkly Rep 2016;65:993-998. DOI: http://dx.doi.org/10.15585/mmwr.mm6537a2

Buchner DM, Cress ME, de Lateur BJ, Esselman PC, Margherita AJ, Price R, Wagner EH. A comparison of the effects of three types of endurance training on balance and other fall risk factors in older adults. Aging (Milano). 1997 Feb-Apr;9(1-2):112-9. PubMed PMID: 9177594.

Centers for Disease Control and Prevention, National Center for Injury Prevention and Control. Web-based Injury Statistics Query and Reporting System (WISQARS) [online]. https://www.cdc.gov/injury/wisqars/index.html(Accessed Aug. 5, 2016).

Florence CS, Bergen G, Atherly A, Burns ER, Stevens JA, Drake C. Medical Costs of Fatal and Nonfatal Falls in Older Adults. Journal of the American Geriatrics Society, March 2018, DOI: 10.1111/jgs.15304

Gerards MHG^{1,2}, McCrum C^{1,3}, Mansfield A ^{4,5,6,7}, Meijer K¹. Perturbation-based balance training for falls reduction among older adults: Current evidence and implications for clinical practice. Geriatr Gerontol Int. December 2017;17(12):2294-2303. doi: 10.1111/ggi.13082. Epub Jun. 16, 2017.

Gillespie LD, Robertson MC, Gillespie WJ, Sherrington C, Gates S, Clemson LM ,Lamb SE. Interventions for preventing falls in older people living in the community. Cochrane Database SystRev. Sep. 12, 2012;(9):CD007146.

Harvey S, Rissel C, Pijnappels M. Associations Between Bicycling and Reduced Fall-Related Physical Performance in Older Adults. J Aging Phys Act. Jul. 1, 2018;26(3):514-519. doi: 10.1123/japa.2017-0243. Epub Jun. 22, 2018. PubMed PMID: 29182418.

Howe TE, Rochester L, Neil F, Skelton DA, Ballinger C. Exercise for improving balance in older people. Cochrane Database Syst Rev. Nov. 9, 2011;(11):CD004963.

Maki BE, McIlroy WE. The role of limb movements in maintaining upright stance: the “change-in-support” strategy. Phys Ther. May 1997;77(5):488-507.

Mansfield A, Wong JS, Bryce J, Knorr S, Patterson KK. Does perturbation-based balance training prevent falls? Systematic review and meta-analysis of preliminary randomized controlled trials. Phys Ther. 2015; 95(5): 700-9.

Mary E. Tinetti, Mark S, Sandra F. Ginter, R.N. Risk Factors for Falls among Elderly Persons Living in the Community N Engl J Med 1988; 319:1701-1707.

McCrum C, Gerards MHG, Karamanidis K, Zijlstra W, Meijer K. A systematic review of gait perturbation paradigms for improving reactive stepping responses and falls risk among healthy older adults. Eur Rev Aging Phys Act. Mar. 2, 2017;14:3. doi: 10.1186/s11556-017-0173-7. eCollection 2017. Review.

Nadeau A, Lungu O, Duchesne C, Robillard MÈ, Bore A, Bobeuf F, Plamondon R, Lafontaine AL, Gheysen F, Bherer L, Doyon J. A 12-Week Cycling Training Regimen Improves Gait and Executive Functions Concomitantly in People with Parkinson’s Disease. Front Hum Neurosci. Jan. 12, 2017;10:690.

Nashner LM: Adapting reflexes controlling the human posture. Exp Brain Res 1976, 26:59-72.

Nashner LM: Balance adjustments of humans perturbed while walking. J Neurophys 1980, 44(4):650-663.

Nashner LM: Fixed patterns of rapid postural responses among leg muscles during stance. Exp Brain Res 1977, 30(1):13-24.

National Institute on Aging. Growing Older in America: The Health and Retirement Study. Washington, DC: U.S. Department of Health and Human Services, 2007.

Okubo Y, Schoene D, Lord SR. Step training improves reaction time, gait and balance and reduces falls in older people: a systematic review and meta-analysis. Br J Sports Med. 2016; 0: 1-9.

Rissel, C., Passmore, E., Mason, C., & Merom, D. (2013). Two pilot studies of the effect of bicycling on balance and leg strength among older adults. Journal of Environmental and Public Health, 2013, 1-6. PubMed ID: 23690805 doi: 10.1155/2013/686412

Rubenstein, L.Z. (2006). Falls in older people: Epidemiology, risk factors and strategies for prevention. Age and Ageing, 35 Suppl 2, ii37-ii41. doi:35/suppl_2/ii37 [pii]

Shani G¹, Shapiro A², Oded G¹, Dima K¹, Melzer I³. Validity of the microsoft kinect system in assessment of compensatory stepping behavior during standing and treadmill walking. Eur Rev Aging Phys Act. 2017 Mar 7;14:4. doi: 10.1186/s11556-017-0172-8. eCollection 2017.

Sherrington C, Whitney JC, Lord SR, Herbert RD, Cumming RG, Close JC. Effective exercise for the prevention of falls: a systematic review and meta-analysis. J Am Geriatr Soc. December 2008;56(12):2234-43.

Smart NA, Dieberg G, Giallauria F. Functional electrical stimulation for chronic heart failure: a meta-analysis. Int J Cardiol. Jul. 15, 2013;167(1):80-6.

Stevens JA. Falls among older adults—risk factors and prevention strategies. J Saf Res. 2005;36(4):409-11.

Verney J, Kadi F, Saafi MA, Piehl-Aulin K, Denis C. Combined lower body endurance and upper body resistance training improves performance and health parameters in healthy active elderly. Eur J Appl Physiol. June 2006;97(3):288-97.

Schmidt, R. A. (1988). Motor Control and Learning: A Behavioral Emphasis, 2^{nd} Edn. Champaign, IL: Human Kinetics.

Schmidt, R. A., and Wrisberg, C. A. (2008). Motor Learning and Performance: A Situation-Based Learning Approach, 4th Edn. Champaign, IL: Human Kinetics.

Schmidt, R. A., and Lee, T. D. (2005). Motor Control and Learning: A Behavioral Emphasis, 4th Edn. Champaign, IL: Human Kinetics.

1-48. (canceled)

49. A mechatronic bicycle simulator system for stimulating balance control of a subject, the system comprising:
a stationary training bicycle (STB);
a moving platform;
one or more motors; and
a central control unit;
wherein the moving platform is configured to provide external perturbation tilts to the STB, thereby stimulating balance control of a subject situated on the STB.

50. The system according to claim 49, wherein the STB comprises a pedal unit and a seat, wherein the relative position of the pedal unit and the seat is adjustable.

51. The system according to claim 50, wherein the pedal unit is adjustable in height, resistance, force, tension and/or speed; and/or wherein the seat is adjustable in height and/or angle.

52. The system according to claim 49, comprising a harness configured to secure the subject during the perturbations.

53. The system according to claim 49, wherein the moving platform is mounted on an axis and is configured to be in a fixed state or a floating state, wherein the moving platform is configured to provide intrinsic self-induced perturbations, when the moving platform is in a floating state.

54. The system according to claim 53, wherein the floating state of the moving platform is configured to be engaged during a time period between external perturbation tilts.

55. The system according to claim 49, further comprising a gear mechanism.

56. The system according to claim 49, further comprising a motion control unit and/or a motion capture unit.

57. The system according to claim 49, wherein the external perturbation tilts are selected from: lateral perturbations (left and right tilt perturbations), Antero-posterior perturbations (forward and backward tilt perturbations), Vertical perturbations, Rotations around a vertical axis, or any combination thereof.

58. The system according to claim 57, wherein the lateral perturbations are in the range of about 0°-20° to each side and/or wherein velocity of the lateral perturbations is in the range of about 0-30 degree/sec and/or wherein acceleration or declaration is in the range of about 0-30 degree/sec²; and/or wherein the antero-posterior perturbations are in the range of about 0°-15 ° to each direction and/or wherein velocity of the antero-posterior perturbations is in the range of about 0-30 degree/sec and/or wherein acceleration or declaration is in the range of about 0-30 degree/sec²; and/or
wherein the vertical perturbations are in the range of about 0-15 cm in each direction, and/or wherein velocity of the vertical perturbations is in the range of about 0-40 cm/sec and/or wherein acceleration or declaration is in the range of about 0-40 cm/sec; and/or
wherein the frequency is in the range of about 1-10 perturbations/min; and/or wherein the rotation perturbations are in the range of about 0-10 cm in each direction, and/or wherein velocity of the rotation perturbations is in the range of about 0-40 cm/sec and/or wherein acceleration or declaration is in the range of about 0-40 cm/sec² and/or wherein the frequency is in the range of about 1-10 perturbations/min.

59. The system according to claim 49, wherein the central control unit is further configured to provide a cognitive challenge to the subject, during at least a portion of a perturbation system.

60. The system according to claim 49, wherein the central control unit comprises a processing unit configured to execute a computer program configured to determine performance of the subject during perturbations and/or determine further perturbations training sessions.

61. The system according to claim 60, wherein the determination of the performance of the subject during perturbations is performed in real time, based at least in part on data related to balance reactive response performance of the subject during perturbations and optionally, during the cognitive tasks; and/or
wherein the determination of further perturbations training sessions is based at least in part on data related to the performance of the subject during a previous perturbation session.

62. The system according to claim 49, wherein the external perturbations are provided in a triangular motion profile for each perturbation; and/or wherein one or more of the provided external perturbations are unexpected.

63. The system according to claim 49, wherein operating parameters of a training session comprise: type of perturbation, maximum acceleration/deceleration of a perturbation, maximum velocity of a perturbation, magnitude of a perturbation, angle of perturbation, number of perturbation repetitions, the delay time between the perturbations, relative position between the seat and the pedal unit, operating parameters of the pedal unit, or any combination thereof.

64. The system according to claim 49, further comprising adjustable gripping handles, comprising a heart rate sensor and/or a pressure sensor.

65. A method for training or improving balance control of a subject, the method comprising:
providing one or more unexpected external perturbations to a subject using the system of claim 49;
detecting a reactive balance response of the subject to the external perturbations based on data acquired by the motion capture unit of the system;
analyze the detected reactive and proactive balance response of the subject; and
providing a feedback to the subject if the balance response is determined to be above a threshold.

66. The method according to claim 65, further comprising providing a cognitive challenge to the subject and determine a cognitive performance of the subject, based on the response to the cognitive challenge; and/or wherein the cognitive challenge is provided in synchronization with an unexpected external perturbation.

67. The method according claim 65, wherein the reactive balance response threshold is customized to the subject, wherein the reactive balance response threshold is determined based on calibration and/or previous training sessions.

68. The method according to claim 65, wherein the analysis of the detected balance response and/or cognitive performance is performed by a computer program comprising one or more machine learning algorithms; and/or
wherein the computer program is configured to provide feedback to the subject indicative of the performance of the subject in the reactive balance response and/or the cognitive challenge and/or wherein the computer program is further configured to adjust operating parameters of the training session based at least in part on the analyzed reactive balance response.

69. A computer-readable storage medium having stored therein machine learning software, executable by one or more processors for executing the method according to claim 65.
